(19) United States
(12) Patent Application Publication
(10) Pub. No.: US 2004/0146948 A1
(43) Pub. Date: Jul. 29, 2004

(54) Compositions and methods for targeting antigen-presenting cells with antibody single-chain variable region fragments

(75) Inventors: Warwick Britton, Bardwell Park (AU); Caroline Demangel, Paris (FR)

Correspondence Address: SPECKMAN LAW GROUP PLLC, 1501 WESTERN AVE, SEATTLE, WA, 98101, US

(73) Assignee: CENTENARY INSTITUTE OF CANCER MEDICINE AND CELL BIOLOGY, Camperdown (AU)

(21) Appl. No.: 10/689,921

(22) Filed: Oct. 17, 2003

Related U.S. Application Data
(60) Provisional application No. 60/420,232, filed on Oct. 18, 2002.

(51) Int. Cl. G01N 33/53; G01N 33/567; C07K 16/28

(52) U.S. Cl. 435/7.2; 530/391.1; 530/388.22

(57) ABSTRACT

Provided are single-chain Fv (scFv) fragment-based compositions and methods for targeting antigens to antigen-presenting cells (APCs) such as, for example, dendritic cells (DC). Compositions and methods disclosed herein are useful in the treatment of diseases including infectious diseases and cancer.

### CROSS-REFERENCE TO RELATED APPLICATIONS

This application claims priority to U.S. Provisional Patent Application No. 60/420,232, filed Oct. 18, 2002.

### BACKGROUND OF THE INVENTION

1. Technical Field of the Invention

The present invention relates generally to the fields of immunology and molecular biology. More specifically, the present invention is directed to antibody single-chain variable region fragment (scFv)-based compositions and methods for targeting antigens to antigen-presenting cells (APCs) such as, for example, dendritic cells (DCs). Compositions and methods disclosed herein are useful in the treatment of diseases including infectious diseases and cancers.

2. Description of the Related Art

Immunization has proved one of the most cost effective strategies for the improvement of human health. Most of the effective vaccines against bacterial, parasitic, and viral pathogens depend on the production of antibodies. Protective immunity against a number of important human and veterinary pathogens depends, however, upon the development of cellular immune responses. In addition, application of therapeutic and prophylactic immunization methodology to vaccines directed against cancers also depends upon the stimulation of cellular immune responses to vaccine components. Accordingly, effective strategies for eliciting cellular immunity will prove widely applicable to the development of vaccines against infectious diseases and cancers.

The in vivo processes involved in the development of cellular immunity continue to be more clearly delineated. One class of antigen-presenting cell, the dendritic cell (DC), is critical in sensing the presence of foreign organisms that play a central role in the induction of antimicrobial immunity. Scattered throughout the body, they constitute the first line of defence against invading pathogens. Innate immune recognition by DCs is based on the recognition of microbial motifs by specialised receptors, the identification of which is a field of growing interest. Moll, Cellular Microbiology 5:493-500 (2003); Figdor et al., Nature Reviews Immunology 2:77-84 (2002); and Demangel et al., Immunology & Cell Biology 78:318-324 (2000). Following interaction with antigen, DC undergo a maturation process resulting in the up-regulation of expression of co-stimulatory, adhesion and MHC molecules enhancing their capacity to present peptides to naïve T cells.

DCs migrate to specialized lymphoid organs, the lymph nodes, to stimulate immunity and undergo maturation to become effective antigen-presenting cells capable of stimulating T lymphocytes (T-cells). This process has been studied in mycobacterial infections such as TB. Infection of DC by M. tuberculosis or BCG induces the co-ordinate processes of DC maturation and secretion of the cytokine interleukin 12 (IL-12). These events are critical in the development of mycobacteria-specific T-cells.

DCs represent a minor cell subset of the peripheral tissues. In steady state conditions, lung DCs constitute less than 1% of the total cell population, a low incidence rate considering their sentinel role against incoming pathogens. Moll et al., Cellular Microbiology 5:493-500 (2003). This sparse distribution is compensated for by a high sensitivity to environmental signals, delivered by damaged endogenous tissues or by pathogens. Austyn, Nature Medicine 5:1232-3 (1999). Microbial products (cell wall components, non-methylated CpG motifs, double stranded RNA) are potent inducers of DC activation. Sousa et al., Current Opinion in Immunology 11:392-399 (1999). Moreover, model antigens expressed in recombinant bacteria are presented by MHC Class I and Class II molecules on DC much more efficiently than the same antigens in soluble form. Svensson et al., J. Immunol. 158:4229-36 (1997); Rescigno et al., Proc. Natl. Acad. Sci. USA 95:5229-34 (1998). This strongly suggests that enhanced antigen presentation could be achieved by selective targeting of subunit vaccines to the DC receptors, which are specialized in the recognition of bacterial products.

Immature DCs display receptors on their surface membranes that permit them to bind to and, in some cases, internalize a diverse array of antigens. Internalized protein-antigens are processed into short peptides that are presented in the context of MHC Class I and Class II molecules. Following the interaction of DC receptors with antigens, DC undergo a maturation that results in the increased expression of co-stimulatory and MHC molecules that enhance their capacity to present peptides to naïve T-cells.

A number of receptor molecules have been identified on the surface of DCs. DEC-205, a homologue of the macrophage mannose receptor, and the integrin CD11c are surface receptors that are restricted to DCs. Immunological evidence suggests that targeting antigens to DEC-205 or to CD11c may improve antigen presentation by DCs. Thus, it is likely that DEC-205 and CD11c play an important role in antigen capture. Rat antibodies directed to mouse DEC-205 are more efficiently internalized than non-specific rat antibodies and are 100-fold more effective at generating T-cell responses to the anti-DEC-205 antibody than to the non-specific rat antibodies. Jiang et al., Nature 375:151-5 (1995). Similar results have been obtained by immunizing mice with anti-CD11c antibodies.

The β2 integrin CD11c is an attractive candidate for investigating the impact of antigen targeting to DCs because it is a DC-restricted surface molecule expressed by all subsets of mouse DCs and all human DCs of myeloid origin. Wilson et al., Immunology and Cell Biology 81:239-246 (2003); and Pulendran et al., Trends in Immunology 22:41-7 (2001). Although its function is still unclear, there is immunological evidence that CD11c is involved in antigen capture and delivery to antigen processing compartments. Finkelman et al., J. Immunol. 157:1406-1414 (1996). So is DEC-205, a lectin receptor expressed by mouse DC subpopulations of the spleen, Peyer's patches, lymph nodes and skin, and by some human DC subsets. Anjuere et al., Blood 93:590-8 (1999) and Guo et al., Human Immunology 61:729-738 (2000). Despite significant sequence homology with the macrophage mannose receptor (MMR) and the presence of eight C-type carbohydrate recognition domains, DEC-205 does not bind mannose and its specific ligands have yet to be defined. Jiang et al., Nature 375:151-5 (1995). Both MMR and DEC-205 receptors mediate adsorptive uptake of antigen in coated vesicles, direct antigen loaded vesicles to the endosomal compartment end recycle to the cell surface. However, whereas MMR recycles through early endosomes, DEC-205 targets antigens to the MHC Class II rich late endosomal compartment, leading to enhanced antigen presentation to CD4⁺ T cells. Guo et al., Human Immunol. 61:729-738 (2000). Improving the delivery of antigens to DEC205 or CD11c receptors may thus result in enhanced T cell priming by DC.

Antigen targeting to sites of immune induction is an efficient means of enhancing immune responses to DNA vaccines. Directing antigens to B7-expressing cells using cytotoxic T-lymphocyte antigen-4 (CTLA4) promotes the development of immune responses to fusion antigen in mice. Boyle et al., Nature 392:408-11 (1998). B7 molecules are expressed by a broad spectrum of leukocytes, including professional antigen presenting cells such as DCs, but also B and T lymphocytes. Products fused to L-selectin, a lymphocyte surface molecule mediating cell entry in the lymph nodes, are less efficient than the CTLA-4 fused ones in promoting T-cell proliferative responses suggesting that selective antigen targeting to cell subsets specialised in antigen presentation is more effective for immune stimulation. The stimulatory effect of scNLDC may also relate to the fact that DEC-205-endocytosis pathway is highly efficient for antigen presentation to CD4⁺ T cells. Mahnke et al., J. Cell Biol. 151:673-683 (2000).

Protein antigen targeting to DEC-205 using chemically-coupled antibody molecules has been shown to induce T cell unresponsiveness in vivo under steady state conditions. Hawiger et al., J. Exp. Med. 194:769-779 (2001) and Bonifaz et al., J. Exp. Med. 196:1627-1638 (2002). Tolerance was, however, converted into prolonged T cell stimulation if the antigen was co-administered with an additional stimulus (such as an anti-CD40 antagonist).

Tuberculosis (TB) is an intracellular bacterial infection, the control of which is dependent upon cellular immunity. TB remains the single most prevalent bacterial infection world-wide, with one third of the world's population currently being infected with Mycobacterium tuberculosis. From this pool of 2 billion infected individuals, 8-9 million new cases of clinical tuberculosis develop a year resulting in the death of at least 2 million people. Because of the interaction of M. tuberculosis and HIV, about half the deaths associated with HIV/AIDS in developing countries occur because of active tuberculosis. The meta-analysis of clinical trials with the only currently available vaccine, M. bovis Bacille Calmette Guerin (BCG), has led to the conclusion that BCG confers about 50% protective efficacy against the common pulmonary form of tuberculosis. This level of efficacy has proven insufficient to control the spread of tuberculosis and underscores the need for new immunization strategies.

Despite the progress that has been made in identifying receptors and other molecules on the surface of APCs and DCs, there remains a need in the art for improved compositions and methods for the delivery of antigens to APCs and DCs in order to achieve improved therapeutic and prophylactic efficacy against diseases including infectious diseases, autoimmune diseases, and cancers.

### SUMMARY OF THE INVENTION

The present invention addresses these and other related needs by providing, inter alia, compositions and methods for targeting antigen-presenting and dendritic cells with antigens, including protein-antigens. As disclosed herein, compositions and methods will find utility in the treatment of disease by enhancing the cellular immune response to antigens.

Disclosed herein are single chain antibody fragments (scFvs) from the monoclonal antibodies NLDC-145 and N418, which are directed to DEC-205 and CD11c mouse DC receptors. Exemplary scFv presented herein have the typical structure of scFvs, with the variable domain of the immunoglobulin heavy chain (V_{H}) linked to the light chain one (V_{L}) via a flexible peptide linker in a V_{H}-V_{L} orientation. Nissim et al., EMBO J. 13:692-698 (1994). These scFvs bind to their target receptor comparably to the parental antibodies in vitro. Thus, scFv targeting, as provided herein, is a powerful means for eliciting strong immune responses in vivo.

Within certain embodiments, the present invention provides antibody single-chain variable region fragments (scFv) for targeting antigen-presenting cells (APCs) such as, for example, dendritic cells (DC). scFv presented herein comprise an antibody heavy chain variable region (V_{H}) operably linked to an antibody light chain variable region (V_{L}) wherein the heavy chain variable region and the light chain variable region together or individually form a binding site for specifically binding to a molecule on the surface of an APC and/or a DC. ScFv may comprise a V_{H} region at the amino-terminal end and a V_{L} region at the carboxy-terminal end. Equally suitable are scFv that comprise a V_{L} region at the amino-terminal end and a V_{H} region at the carboxy-terminal end.

An exemplary scFv is derived from monoclonal antibody NLDC-145 which antibody specifically binds to DEC-405 on the surface of DC. According to this embodiment, the scFv comprises variants of the NLDC-145 heavy chain (V_{H}) and light chain (V_{L}) variable regions wherein each variant NLDC-145 heavy chain (V_{H}) and light chain (V_{L}) region is at least 70%, 80%, 90%, 95% or 98% identical to the sequences disclosed herein in SEQ ID NOs: 5 and 6, respectively. A most preferred exemplary scFv, disclosed herein in SEQ ID NO: 7, comprises the NLDC-145 heavy chain (V_{H}) and light chain (V_{L}) variable regions disclosed herein in SEQ ID NOs: 5 and 6, respectively.

An alternative preferred exemplary scFv is derived from monoclonal antibody N418 which antibody specifically binds to CD11c on the surface of DC. According to this embodiment, the scFv comprises variants of the N418 heavy chain (V_{H}) and light chain (V_{L}) variable regions wherein the variant N418 derived scFv is at least 70%, 80%, 90%, 95% or 98% identical to the sequences disclosed herein in SEQ ID NO: 2. A most preferred exemplary scFv comprises the N418 heavy chain (V_{H}) and light chain (V_{L}) variable regions which scFv is disclosed herein in SEQ ID NOs: 2.

ScFv disclosed herein may, optionally, further comprise a polypeptide linker operably linked between the heavy chain variable region and the light chain variable region. Polypeptide linkers of the present invention generally comprise between 1 and 50 amino acids. More common are polypeptide linkers of at least 2 amino acids. Even more commonly, polypeptide linkers are between 3 and 12 amino acids. An exemplary linker peptide for incorporating between scFv heavy and light chains comprises the 5 amino acid sequence Gly-Gly-Gly-Gly-Ser. Alternative exemplary linker peptides comprise one or more tandem repeats of the sequence Gly-Gly-Gly-Gly-Ser to create linkers comprising, for example, the sequences Gly-Gly-Gly-Gly-Ser-Gly-Gly-Gly-Gly-Ser, Gly-Gly-Gly-Gly-Ser-Gly-Gly-Gly-Gly-Ser-Gly-Gly-Gly-Gly-Ser, and Gly-Gly-Gly-Gly-Ser-Gly-Gly-Gly-Gly-Ser-Gly-Gly-Gly-Gly-Ser-Gly-Gly-Gly-Gly-Ser.

Surface molecules on APC and/or DC that may be targeted by scFv of the present invention include proteins and carbohydrates. Within certain embodiments, surface protein molecules include receptor proteins. Surface receptor proteins may facilitate internalization of the specifically bound scFv into the APC and/or the DC. Within certain aspects, specifically bound scFv may be internalized by receptor-mediated endocytosis and/or by pinocytosis. Preferred surface protein molecules include, but are not limited to, the mannose receptor (MR), chemokine receptor 1 (CCR1), B7-1 (CD80), B7-2 (CD86), CD40, CD11c, DEC-205, a Toll-like receptor (TLR), and the Fcγ receptor (FcγR). Most preferred are those surface protein molecules that are restricted to DCs such as CD11c and DEC-205.

Other aspects of the present invention provide complexes between scFv and one or more antigens, including protein-antigens. Antigens encompass protein-antigens that undergo in vivo post-translational modifications wherein the protein-antigen may be glycosylated, lipidated, phosphorylated or the like.

Further aspects of the present invention provide complexes comprising scFv and a lipid. Thus, exemplified herein are scFv-lipid complexes wherein the scFv comprises a tag such as an affinity tag. Suitable affinity tags include, but are not limited to, the FLAG-tag and the hexahistidine tag. Thus, for example, a hexahistidine tagged scFv may form a complex directly with a lipid, such as a metal chelating lipid. An exemplary metal chelating lipid presented herein is nitrilotriacetic acid ditetradecylamine (NTA-DTDA).

Within still further aspects of the present invention, scFv may be complexed directly with a lipid and/or with one or more antigen that is encapsulated by, incorporated within, and/or associated with a lipid membrane, a lipid bi-layer, and/or a lipid complex such as, for example, a liposome, a vesicle, a micelle and/or a microsphere. Thus, within these aspects of the invention, the term “antigen” encompasses such liposomes, vesicles, micelles and/or microspheres that comprise an antigen, such as a protein-antigen, including glycoprotein-antigens and/or lipoprotein-antigens.

Complexes between scFv, a lipid, and/or an antigen may be achieved by chemical crosslinking or, alternatively, may be a fusion protein comprising scFv heavy and light chain variable regions and an antigen. Suitable scFv that may be employed in the complexes comprising an scFv, such as scFv/antigen, scFv/lipid, and scFv/lipid/antigen complexes, include those indicated above and as described in further detail herein below. scFv/antigen complexes are capable of specifically binding to APC and/or DC thereby facilitating the targeting of the antigen to the APC and/or DC.

An exemplary scFv/antigen complex presented herein is the scFv NLDC-145-85B encoded by the nucleotide sequence presented herein as SEQ ID NO: 8. Equally preferred are functional fragments, derivatives and variants of the scFv NLDC-145-85B encoded by the nucleotide sequence presented herein as SEQ ID NO: 8. Functional variants of scFv NLDC-145-85B preferably exhibit at least about 70%, more preferably at least about 80% or 90% and most preferably at least about 95% or 98% sequence identity to the polypeptide encoded by SEQ ID NO: 8.

Another exemplary scFv/antigen complex presented herein is the scFv N418-85B encoded by the nucleotide sequence presented herein as SEQ ID NO: 3. Equally preferred are functional fragments, derivatives and variants of the scFv N418-85B encoded by the nucleotide sequence presented herein as SEQ ID NO: 3. Functional variants of scFv N418-85B typically exhibit at least about 70%, more typically at least about 80% or 90% and most typically at least about 95% or 98% sequence identity to the polypeptide encoded by SEQ ID NO: 3.

Within certain aspects, antigens that may be complexed with the inventive scFv include protein-antigens from an organism, including a virus, parasite or a bacterium, which is capable of causing an infectious disease in a human. Exemplary viral organisms include, but are not limited to, human immunodeficiency virus (HIV), a herpes virus, and an influenza virus. Exemplary parasitic organisms include, but are not limited to, Leishmania (e.g., L. major and L. donovani). Exemplary bacterial organisms include, but are not limited to, Mycobacteria (e.g., M. tuberculosis and M. bovis), Chlamydia (e.g., C. trachomatis and C. pneumoniae), and Ehrlichia (e.g., E. sennetsu, E. chaffeensis, E. ewingii, and E. phagocytophila). Within certain aspects, the protein-antigen is an M. tuberculosis antigen selected from the group consisting of 85B, MPT64, and ESAT-6 disclosed herein in SEQ ID NO: 14, SEQ ID NO: 16, and SEQ ID NO: 18, respectively. Other aspects provide that the protein-antigen is a fragment, derivative or variant of 85B, MPT64, or ESAT-6. Typical protein-antigens exhibit at least about 70%, more typically at least about 80% or 90% and most typically at least about 95% or 98% sequence identity to the polypeptide disclosed herein in SEQ ID NO: 14, SEQ ID NO: 16, and/or SEQ ID NO: 18.

The present invention also provides fusion proteins, comprising an antigen-presenting cell binding protein and a protein-antigen wherein the fusion protein is capable of specifically binding to an antigen-presenting cell (APC) and/or a dendritic cell (DC) and in inducing a protein-antigen specific T-cell response. According to certain aspects, the APC and/or DC binding protein specifically binds to a receptor on the APC and/or DC. Exemplary receptors include, but are not limited to, the mannose receptor (MR), chemokine receptor 1 (CCR1), B7-1, B7-2, CD40, CD11c, DEC-205, a Toll-like receptor (TLR), and the Fcγ receptor (FcγR). Preferred antigens are infectious disease antigens, autoimmune disease antigens, or cancer cell antigens, including tissue-specific and/or tumor-specific antigens, as indicated above and as described in further detail herein.

Further aspects of the present invention provide polynucleotides that encode one or more of the scFv presented herein. Within certain embodiments, the polynucleotide is a component of a vector, such as a plasmid vector or a viral vector, wherein the vector comprises a transcriptional promoter operably linked to the scFv encoding polynucleotide.

Related aspects provide polynucleotides that encode an s cFv/antigen fusion protein which polynucleotides comprise a first polynucleotide that encodes an scFv and a second polynucleotide that encodes one or more protein-antigen wherein the first polynucleotide and the second polynucleotide are operably linked such that together they encode a fusion protein comprising an scFv and a protein-antigen. More preferred embodiments provide that the first polynucleotide and the second polynucleotide are operably linked by a third polynucleotide that encodes a polypeptide linker between the scFv and the protein-antigen. Within certain embodiments, the polynucleotide encoding the scFv/protein antigen fusion protein is a component of a vector, such as a plasmid vector or a viral vector, wherein the vector comprises a transcriptional promoter operably linked to the s cFv encoding polynucleotide. Particularly preferred vectors comprising a polynucleotide encoding an scFv and an scFv/antigen are, respectively, the pcDNA3-NLDC-145 and pcDNA3-NLDC-85 plasmid vectors presented herein in FIG. 1 as well as pcDNA3-N418-85. The nucleotide sequences encoding scFv NLDC-145-85B, scFv N418-85B, and the nucleotide sequence of pcDNA3 are presented herein in SEQ ID NO: 8, SEQ ID NO: 3, and SEQ ID NO: 9, respectively.

The present invention also provides compositions comprising scFv, scFv/lipid, scFv/antigen, and/or scFv/lipid/antigen complexes as well as compositions comprising polynucleotides encoding scFv and/or scFv/antigen complexes and compositions comprising vectors comprising one or more polynucleotides encoding an scFv and/or an scFv/antigen complex. Exemplary compositions may, optionally, further comprise a cytokine such as interleukin-12 (IL-12), IL-6, IL-4, IL-1, interferon-γ (IFNγ), GM-CSF, tumor necrosis factor (TNF), and/or the CD40 ligand CD154, and/or may comprise a lipopolysaccharide (LPS) or other inducer of the DC response to antigen, such as other cell wall components, non-methylated CpG motifs, and/or double-stranded RNA.

Other aspects of the present invention provide methods for introducing an antigen into an antigen-presenting cell (APC) and/or a dendritic cell (DC), the methods comprising the steps of: (a) isolating from a patient sample, an APC and/or a DC; and (b) contacting the APC and/or DC with an scFv/antigen complex, wherein the scFv/antigen complex is in contact with the APC and/or DC under conditions and for such a time as required to permit the antigen to enter the APC and/or DC.

Related aspects of the present invention provide methods for introducing an antigen into an APC and/or a DC of a patient, the methods comprising the step of administering to a patient a composition comprising an scFv/antigen complex, thereby inducing an interaction with an APC and/or a DC of the patient.

Still further related aspects provide methods for introducing a protein-antigen into an APC and/or a DC of a patient, the methods comprising the step of administering to the patient a composition comprising a polynucleotide encoding an scFv/antigen complex.

Still further aspects of the present invention provide methods for treating a disease and/or modulating an immune response in a patient, the methods comprising the steps of: (a) obtaining from the patient a sample comprising an antigen-presenting cell (APC) and/or a dendritic cell (DC); (b) contacting the sample with an scFv/antigen complex under conditions and for such a time as required to allow binding of the scFv/antigen complex to the APC and/or DC; and (c) administering the scFv/antigen APC and/or DC-bound complex to the patient. Modulation of the immune response may include enhancing, stimulating, suppressing, and/or blocking the immune response in the patient.

Within methods for the present invention, the disease may be selected from the group consisting of an infectious disease, an autoimmune disease and a cancer. More preferred methods provide that the infectious disease is caused by an organism selected from the group consisting of Leishmania, Mycobacteria, Chlamydia, and Ehrlichia. Equally preferred methods provide that the cancer is selected from the group consisting of soft tissue sarcomas, lymphomas, and cancers of the brain, esophagus, uterine cervix, bone, lung, endometrium, bladder, breast, larynx, colon/rectum, stomach, ovary, pancreas, adrenal gland and prostate.

Other aspects provide methods for inhibiting, reducing, suppressing and/or blocking the activity of a target antigen on the surface of an antigen-presenting cell (APC) and/or a dendritic cell (DC), the methods comprising the steps of: (a) obtaining a sample comprising and APC and/or a DC; (b) contacting the APC and/or DC with an scFv capable of specifically binding to the target antigen on the surface of the APC and/or DC under conditions and for such a time as required to permit binding of the scFv to the APC and/or DC, wherein binding of the scFv to the APC and/or DC blocks or substantially reduces the activity of the target antigen, thereby inhibiting, reducing, suppressing and/or blocking an immune response.

By any of the methods disclosed herein, the scFv may bind to a molecule, including a carbohydrate molecule or a protein molecule, on the surface of the APC and/or DC. Preferred surface protein molecules include, but are not limited to, the mannose receptor (MR), chemokine receptor 1 (CCR1), B7-1, B7-2, CD40, CD11c, DEC-205, a Toll-like receptor (TLR), and the Fcγ receptor (FcγR).

Within certain methods, the scFv may be complexed to an antigen wherein scFv/antigen complexes are achieved by chemical crosslinking or wherein scFv/antigen complexes are scFv/antigen fusion proteins.

Suitable antigens that may be employed in any of the methods disclosed herein include, but are not limited to, antigens from an organism, including a virus, a parasite, or a bacterium, which is capable of causing an infectious disease in a human. Exemplary viral organisms include, but are not limited to, human immunodeficiency virus (HIV), a herpes virus, and an influenza virus. Exemplary bacterial organisms include, but are not limited to, Mycobacteria, Chlamydia, and Ehrlichia. Exemplary parasitic organisms include, but are not limited to, Leishmania. Within certain aspects, the antigen is an M. tuberculosis antigen selected from the group consisting of 85B, MPT64, and ESAT-6 disclosed herein in SEQ ID NO: 14, SEQ ID NO: 16, and SEQ ID NO: 18, respectively.

These and other aspects of the present invention will become apparent upon reference to the following detailed description and attached drawings. All references disclosed herein are hereby incorporated by reference in their entirety as if each was incorporated individually.

### BRIEF DESCRIPTION OF THE DRAWINGS AND SEQUENCE IDENTIFIERS

FIG. 1 depicts plasmid maps for (A) pcDNA3-NLDC-145 expressing an scFv derived from anti-DEC-205 hybridoma NLDC-145, and (B) pcDNA3-NLDC-85 in which the gene for the M. tuberculosis antigen 85B is fused to the anti-DEC-205 derived scFv.

FIG. 2 depicts a plasmid map of DNA vectors used for transfections and immunizations exemplified within the examples disclosed herein. Two vectors were constructed in which the ScNLDC or ScN418 sequences were fused to the Ag85B gene via a 12 amino acid spacer. The scFv-Ag85B construct was linked 5′ to HBM secretion sequence and 3′ to a FLAG detection sequence (i.e. Asp-Tyr-Lys-Asp-Asp-Asp-Asp-Lys). Transcription of the fusion protein in mammalian cells was under the control of the human cytomegalovirus promoter (PCMV) and the bovine growth hormone transcription terminator BGHpA.

FIG. 3A depicts the deduced amino acid sequence of V_{H} and V_{L} domains in NLDC-145 and N418 monoclonal antibodies. Underlined sequences correspond to complementarity determining regions (CDR). FIG. 3B presents the results of an analysis of culture supernatant from insect cells infected with recombinant baculoviruses expressing ScNLDC: Coomassie blue staining (1), Western blot using detection antibodies binding FLAG (2) and polyHis (3) peptides.

FIGS. 4A-4D present immunohistological data demonstrating that scNLDC binds Langerhans cells with the same specificity as the parental antibody. Epidermal sheets of mouse ears stained with NLDC-145 whole IgG molecule (A) or with purified ScNLDC, as detected via the C-terminal poly-Histidine tail (B) or FLAG peptide (D), were compared. A control epidermis incubated with secondary reagents in the absence of ScNLDC is shown (C).

FIGS. 5A-5D present immunohistological data demonstrating that scN418 binds dendritic cells with the same specificity as the parental antibody. FSDCs stained with N418 whole IgG molecule (A) or with purified ScN418, as detected via the C-terminal poly-Histidine tail (B) or FLAG peptide (D), were compared. Control cells incubated with secondary reagents only were included (C).

FIG. 6 is a bar graph depicting induction of an Interferon-gamma (IFN-γ) T-cell response following in vivo administration of the pscNLDC-Ag85B, pAg85B, or the control vector pCDNA3 (ctrl). Mice were compared for the frequency of IFN-γ producing cells in the spleen (A) and total IFN-γ production (B) following restimulation with purified Ag85B protein four weeks after single injection of DNA. Mean (±SE) in three mice groups are shown, and are representative of three independent experiments. Differences between groups were analyzed using ANOVA (*p<0.05, **p<0.01).

FIG. 7 is a bar graph depicting induction of specific antibody response following in vivo administration of mice with the pcDNA3-NLDC-85 and pcDNA3-85 vectors. Titers of Ag85B-specific serum IgG were compared in mice immunized with pscNLDC-Ag85B, pAg85B or the control vector pCDNA3 (ctrl), two and four weeks after injection of a single dose of DNA vaccine. The horizontal dotted line indicates the background level of the ELISA. Mean (±SE) in three mice groups are shown, and are representative of two independent experiments.

FIG. 8 is a bar graph depicting the protective effect of immunization of mice with pcDNA3-NLDC-85, pcDNA3-85 and the control pcDNA3 vectors and the currently used live vaccine BCG. C57BL/6 mice (n=5) were immunised by three intramuscular injections of 100 μg of each of the three DNA vaccines or 5×10⁴ BCG by subcutaneous injection. The bacterial counts of M. tuberculosis (mean ±SD) in the lungs and spleens of mice (n=5) were determined 4 weeks after aerosol infection with M. tuberculosis. The pcDNA3-NLDC-85 vaccine was significantly more effective than pcDNA-85 vaccine (p<0.05) and the control pcDNA3 vaccine (p<0.01) and there was no significant difference in the effect of the pcDNA3-NLDC-85 vaccine and BCG.

SEQ ID NO: 1 is the nucleotide sequence encoding scFv N418 of SEQ ID NO: 2.

SEQ ID NO: 2 is the amino acid sequence of scFv N418.

SEQ ID NO: 3 is the nucleotide sequence for scFv N418-85B.

SEQ ID NO: 4 is the nucleotide sequence encoding scFv NLDC145 of SEQ ID NO: 7.

SEQ ID NO: 5 is the deduced amino acid sequence of the heavy chain variable region (V_{H}) of the NLDC-145 monoclonal antibody.

SEQ ID NO: 6 is the deduced amino acid sequence of the light chain variable region (V_{L}) of the NLDC-145 monoclonal antibody.

SEQ ID NO: 7 is the amino acid sequence of scFv NLDC145.

SEQ ID NO: 8 is the nucleotide sequence for scFv NLDC-145-85B.

SEQ ID NO: 9 is the nucleotide sequence for pcDNA3 (Invitrogen; Carlsbad, Calif.).

SEQ ID NO: 10 is the amino acid sequence of an exemplary linker peptide for incorporating between an scFv and an antigen in an scFv/antigen complex.

SEQ ID NO: 11 is the nucleotide sequence encoding the linker peptide of SEQ ID NO: 10.

SEQ ID NO: 12 is the nucleotide sequence for baculovirus vector pBACPak 8 (Genbank Accession No. U02446).

SEQ ID NO: 13 is the nucleotide sequence encoding M. tuberculosis antigen 85B (Genbank Accession No. X62398).

SEQ ID NO: 14 is the amino acid sequence for M. tuberculosis antigen 85B (Genbank Accession No. CAA44269).

SEQ ID NO: 15 is the nucleotide sequence encoding M. tuberculosis antigen mpt64 (Genbank Accession No. X75361).

SEQ ID NO: 16 is the amino acid sequence for M. tuberculosis (H37Rv) antigen MPT64 (Genbank Accession No. NP_{—}216496).

SEQ ID NO: 17 is the nucleotide sequence encoding M. tuberculosis antigen esat-6 (Genbank Accession No. AF420491).

SEQ ID NO: 18 is the amino acid sequence for M. tuberculosis antigen ESAT-6 (Genbank Accession No. Q57165).

### DETAILED DESCRIPTION OF THE INVENTION

As indicated above, the present invention is directed to antibody single-chain variable region fragment (scFv)-based compositions and methods for targeting antigen-presenting cells (APCs) such as, for example, dendritic cells (DC). Disclosed herein are scFv-based complexes, such as scFv/lipid, scFv/antigen, and scFv/lipid/antigen complexes, which specifically bind to molecules on the surface of APC and/or DC and, in the case of scFv/antigen complexes, are suitable for introducing the antigen into the APC and/or DC. Complexes of the present invention may be employed to enhance and/or stimulate T-cell responses to candidate antigen and, as exemplified herein, known antigens of M. tuberculosis such as 85B. Inventive scFv-based complexes may be used to enhance and/or stimulate the immune response in the patient thereby reducing the severity of the infectious diseases, including diseases caused by mycobacterial infections such as tuberculosis.

Also disclosed herein are methods employing the inventive scFv-based complexes which methods are suitable for blocking the activity of a target antigen on the surface of an APC; for introducing antigens, either ex vivo or in vivo, into APC; for modulating, stimulating and/or inhibiting an immune response in a patient; as well as for treating a disease in a patient such as an infectious disease, an autoimmune disease, and a cancer. Without being limited to a particular mode of action, the methods disclosed herein may facilitate T-cell priming for antibody production and may provide an effective mechanism for increasing antibody responses to recombinant protein-antigens. In addition, the c ombined effect of increasing T-cell and antibody responses to antigens may be particularly applicable to tissue-specific and tumor-specific antigens that are associated with cancers.

Each of these aspects of the present invention is described in further detail herein below.

As used in this specification and the appended claims, the singular forms “a,” “an” and “the” include plural references unless the context clearly dictates otherwise.

The practice of the present invention will employ, unless indicated specifically to the contrary, conventional methods for virology, immunology, microbiology, molecular biology and recombinant DNA techniques within the skill of the art, many of which are described below for the purpose of illustration. Such techniques are explained fully in the literature. See, e.g., Sambrook, et al., “Molecular Cloning: A Laboratory Manual” (2nd Edition, 1989); Maniatis et al., “Molecular Cloning: A Laboratory Manual” (1982); “DNA Cloning: A Practical Approach, vol. I & II” (D. Glover, ed.); “Oligonucleotide Synthesis” (N. Gait, ed., 1984); “Nucleic Acid Hybridization” (B. Hames & S. Higgins, eds., 1985); “Transcription and Translation” (B. Hames & S. Higgins, eds., 1984); “Animal Cell Culture” (R. Freshney, ed., 1986); and Perbal, “A Practical Guide to Molecular Cloning” (1984). All publications, patents and patent applications cited herein, whether supra or infra, are hereby incorporated by reference in their entirety.

Antigen-Presenting Cell (APC)- and Dendritic Cell (DC)-specific Molecules

As noted above, the present invention provides single-chain variable region fragment (scFv)-based complexes and compositions and methods employing such complexes for targeting antigen-presenting cells (APCs), including dendritic cells (DCs). Each of the inventive scFv that are disclosed, and employed in the inventive complexes, compositions and methods, specifically bind to a carbohydrate and/or protein molecule on the surface of an APC and/or a DC.

As used herein, the term “specifically bind” or “specifically binding” refers to the ability of an antibody, and/or an scFv derived from that antibody, to detect a target molecule or single epitope out of a population of non-target molecules on the surface of an antigen-presenting cell such as a dendritic cell. Specific binding may be determined by a number of methods available in the art including, for example, assays based on primary interactions between an antibody and/or scFv and the corresponding target molecule. Exemplary assays for measuring primary interactions include radioimmunoassay (RIAs) and enzyme-linked immunosorbent assays (ELISAs). In addition, specific binding may be determined by measuring secondary interactions such as by measuring changes in the physical and/or biochemical properties of the target antigen that occur as a consequence of contacting the target antigen with an antibody and/or an scFv. For example, secondary interactions may be measured by immunoprecipitation of a labeled target antigen followed by detection of the label or by detection of a reaction, such as autophosphorylation, catalyzed by the immunoprecipitated antigen.

The term “antigen-presenting cell” or “APC” refers to those highly specialized cells that can process antigens and display their peptide fragments on the cell surface together with molecules required for lymphocyte activation. The main antigen-presenting cells for T-cells are DC, macrophages, and B-cells, whereas the main antigen-presenting cells for B-cells are follicular dendritic cells.

The term “dendritic cell” or “DC” is defined as those APCs that are found in T-cell areas of lymphoid tissues. Banchereau et al., Nature 392:245-251 (1998). DCs are a sparsely distributed, migratory group of bone-marrow-derived leukocytes that are specialized for the uptake, transport, processing and presentation of antigens to T-cells. Non-lymphoid tissues also contain DCs, but these do not stimulate T-cell responses until they are activated and migrate to lymphoid tissues. In general, dendritic cells may be identified based on their typical shape (stellate in situ, with marked cytoplasmic processes, dendrites, visible in vitro); their ability to take up, process and present antigens with high efficiency; and their ability to activate naïve T-cell responses. DCs of the present invention are distinct from the follicular DC that present antigens to B-cells. For a general review of murine and human dendritic cells, see Shortman et al., Nat. Rev. Immunol. 2(3):151-61 (2002).

Exemplary surface molecules on APC and/or DC that may be targeted by the scFv of the present invention are receptor molecules including, but not limited to, the mannose receptor (MR), chemokine receptor 1 (CCR1), B7-1 (CD80), B7-2 (CD86), CD40, CD11c, DEC-205, a Toll-like receptor (TLR), and the Fcγ receptor (FcγR). Most preferred are those surface protein molecules that are restricted to DCs such as CD11c and DEC-205. Other embodiments of the present invention provide scFv that bind specifically to carbohydrates and/or carbohydrates attached to APC and/or DC-specific surface molecules.

The mannose receptor (MR) is expressed by macrophages and DC and recognizes carbohydrate groups such as mannose or fucose that are exposed on a number of microorganisms, including mycobacteria. Sallusto et al., J. Exp. Med. 182:389-400 (1995); Engering et al., Adv. Exp. Med. Biol. 417:183-7 (1997); Engering et al., Eur. J. Immunol. 27:2417-25 (1997); Prigozy et al., Immunol. 6:187-97 (1997); and Tan et al., Adv. Exp. Med. Biol. 417:171-4 (1997). Mannose receptors on macrophages recycle constitutively and may thus allow antigen internalization in successive rounds. Because ligand binding to MR induces the release of pro-inflammatory cytokines such as IL-1, IL-6 and IL-12 by DC, MR engagement may act by promoting both antigen presentation and DC maturation, and therefore further facilitate T-cell stimulation. Yamamoto et al., Infect. Immun. 65:1077-82 (1997) and Shibata et al., J. Immunol. 159:2462-7 (1997).

CCR1 is the main receptor expressed by immature DC and is downregulated on LPS- or TNF-mediated activation of DC. Because immature DC are more efficient at capturing and processing antigens than are mature DC, it may be advantageous to target scFv/antigens of the present invention to immature DC by utilizing scFv that specifically bind to CCR1.

B7-1 (CD80) and B7-2 (CD86) are co-stimulatory glycoprotein molecules expressed on APC. The B7 molecules are homodimeric members of the immunoglobulin superfamily found exclusively on the surface of cells capable of stimulating T-cell growth. These molecules bind to CD28 on T-cells to co-stimulate the growth of naïve T-cells. CD80 (B7-1) is expressed on monocytes, immature dendritic cells and activated B cells and T cells. It is important in the regulation of T cell activation and is a ligand for CD28 and CD152 (CTLA-4). CD86 (B7-2) is expressed on interdigitating dendritic cells and monocytes, upregulated on recirculating B cells following activation, germinal B cells and memory B cells. CD86 is a coreceptor for CD28 and CD152 (CTLA-4).

CD40 is a transmembrane protein expressed on APC including macrophages and B-cells. CD40 is found on normal and neoplastic B-cells Hodgkin and Reed-Stemberg cells, normal basal epithelial andepithelial cell carcinomas, interdigitating cells (IDC), marcophages, follicular dendritic cells, fibroblasts keratinocytes and some endothelial cells. Ligation of CD40 on B-cells mediates diverse outcomes depending on the stage of differentiation and the epitope engaged. CD40 plays a central role in developing and promoting events associated with T-cell differentiation and antibody responses. Ligation of CD40 on macrophages induces them to secrete TNF-α and to become receptive to reduced concentrations of IFN-γ while ligation of CD40 on B-cells promotes growth and antibody isotype switching.

CD11c is a DC restricted integrin that, similar to DEC-205, induces a strong T-cell immune response when stimulated with anti-CD11c antibodies. Finkelman et al., J. Immunol. 157:1406-14 (1996). CD11c is able to recognize several microbial substances, including bacterial lipopolysaccharide (LPS), the lipophosphoglycan of Leishmania, the filamentous hemagglutinin of Bordetella, and structures on yeasts such as Candida and Histoplasma.

DEC-205 is a macrophage mannose receptor-related C lectin that is restricted in expression to DCs and is involved in antigen processing. Witmer-Pack et al., Cell. Immunol. 163:157-62 (1995); Inaba et al., Cell. Immunol. 163:148-56 (1995); U.S. Pat. No. 6,117,977 and U.S. Pat. No. 6,046,158. The multilectin domain structure of DEC-205 suggests that it may enable DC to bind highly diverse carbohydrate-bearing antigens. Antigen targeting to DEC-205 may improve antigen presentation by DC, indicating the potential for DEC-205 to capture and deliver antigen to processing compartments.

The Toll-like receptors (TLRs) on mammalian cells are able to detect a variety of microbial components and, consequently, are a major component of the innate immunity to microbial infections. Anderson, Curr. Opin. Immunol. 12:13-19 (2000). Mycobacterial lipoproteins appear to stimulate IL-12 on human macrophages through Toll-like receptor 2 (TLR2). Brightbill et al., Science 285:732-6 (1999). TLRs may participate in the induction of primary responses to mycobacteria by DC. Demangel et al., Immunol. and Cell Biol. 78:318-324 (2000).

The FC gamma receptor (FCγR) is expressed on DC. FCγR binds to the constant region of immunoglobulins of the IgG isotype and induces endocytosis of the immune complexes. Cella et al., Curr. Opin. Immunol. 9:10-16 (1997).

Preferred APC- or DC-specific molecules facilitate the specific binding and/or introduction of an inventive scFv, scFv/lipid complex, scFv/antigen complex, and/or scFv/lipid/antigen complex into the APC or DC by a process of internalization such as, for example, receptor-mediated endocytosis or pinocytosis and, most preferably, enable the display of peptides derived from the in vivo-processing of the antigen on the cell-surface within the context of MHC Class I or MHC Class II molecules.

Single-Chain Fv (scFv)

As noted above, the present invention provides antibody single-chain Fv (scFv) scFv-based complexes, including, but not limited to, scFv/lipid, scFv/antigen, and scFv/lipid/antigen complexes wherein the scFv specifically binds to a molecule on the surface of an APC and/or a DC. As used herein, the terms “single-chain Fv” and “scFv” refer to recombinant proteins comprising an antibody heavy chain variable (V_{H}) region operably linked to an antibody light chain variable (V_{L}) region. Optionally, scFv further comprise a “linker” peptide that serves as a spacer between the heavy chain variable region and the light chain variable region. In addition, scFv may comprise a signal (or leader) sequence at the N-terminal end that co-translationally or post-translationally directs transfer of the protein and/or may comprise a tag, such as an affinity tag, exemplified by the FLAG-tag and hexahistidine tag, to facilitate complex formation such as scFv/antigen, scFv/lipid, and/or scFv/lipid/antigen complex formation.

ScFv constructs of the present invention provide numerous advantages over whole antibody-based therapeutics. Because scFvs are produced in substantial quantities and with minimal purification requirements, such as in a baculovirus expression system, they represent an economical alternative to whole antibody molecules. Moreover, scFvs may be administered repeatedly without inducing deleterious host immune responses against the Fc part of the immunoglobulin chains.

A large array of antigens and lipids can be potentially directed to DCs using the scFv constructs of the present invention. For example, protein antigens may be fused to scFvs by genetic engineering methodologies, and potentially any kind of compounds may be chemically joinable to the scFv via its affinity tag.

Single-chain Fv may be generated by a number of methodologies that are readily available in the art. Most commonly, scFv are generated from hybridomas that express a monoclonal antibody having the desired antigen binding specificity and affinity. For example, scFv of the present invention may be generated from hybridomas that express monoclonal antibodies that specifically bind to a molecule that is exposed on the surface of an APC and/or a DC. Exemplified herein are scFv that were generated from hybridomas, designated NLDC-145 and N418, that express monoclonal antibodies that specifically bind to DEC-205 and CD11c, respectively, on the surface of dendritic cells.

Polynucleotides encoding antibody heavy and light chain variable regions may be amplified from total hybridoma cell RNA. For example, first-strand cDNA may be synthesized using reverse transcriptase and random hexamers. Heavy and light chain variable regions may then be amplified from the cDNA by utilizing primer pairs that hybridize 5′ and 3′ to each of the heavy and light chain variable region coding regions. See, for example, U.S. Pat. Nos. 4,683,195, 4,683,202, and 4,800,159. Primer sequences suitable for PCR amplification of scFv heavy and light chains are disclosed in U.S. Pat. No. 6,248,516 and PCT Patent Application Publication No. WO 90/05144.

Polynucleotides isolated in this way may be combined by utilizing conventional recombinant DNA methodology such that the polynucleotide comprising the V_{H} coding region is fused in-frame with the polynucleotide comprising the V_{L} coding region. Depending on the precise scFv to be expressed, it may be desirable to fuse the V_{H} coding region 5′ to the V_{L} coding region. Alternatively, the V_{H} coding region may be fused 3′ to the V_{L} coding region. Regardless of the orientation, in-frame fusion of the V_{H} and V_{L} coding regions permits translation into a single scFv protein that retains the biological activity of the component V_{H} and V_{L} polypeptides. (For general guidance on the design of scFv, see U.S. Pat. No. 4,946,778).

A polynucleotide encoding a peptide linker sequence may be employed to separate the encoded V_{H} and V_{L} regions by a distance sufficient to ensure that each polypeptide folds into a functional secondary and tertiary structure. Such a peptide linker sequence is incorporated into the fusion protein using standard recombinant DNA techniques well known in the art. Suitable peptide linker sequences may be chosen based on the following factors: (1) the ability of the linker to adopt a flexible extended conformation; and (2) the lack of hydrophobic or charged residues that might react with the antigen binding sites on, or created by, the V_{H} and V_{L} regions. Preferred peptide linker sequences contain Gly, Asn and Ser residues. Other near neutral amino acids, such as Thr and Ala may also be used in the linker sequence. A particularly preferred peptide linker exemplified herein comprises three tandem repeats of the five amino acid sequence Gly-Gly-Gly-Gly-Ser to generate the 15 amino acid linker having the amino acid sequence Gly-Gly-Gly-Gly-Ser-Gly-Gly-Gly-Gly-Ser-Gly-Gly-Gly-Gly-Ser.

Depending on the particular application contemplated, it may be desired to design the scFv to favor the formation of a multimer such as a dimer, trimer, and/or tetramer of the monomeric scFv. For example, within the context of tumor targeting, where scFv having molecular weights in the 60-100 kDa range have been shown to exhibit increased tumor penetration and faster clearance rates compared to the parent immunoglobulin, it may be preferred to form scFv dimers (˜60 kDa), trimers (˜90 kDa) or tetramers (˜120 kDa). Kortt et al., Biomol. Eng. 18(3):95-108 (2001).

ScFv multimers may be achieved by varying the length of the polypeptide linker that joins the heavy chain variable region to the light chain variable region. Amino acid sequences that may be usefully employed as linkers include those disclosed in Maratea et al., Gene 40:39-46 (1985); Murphy et al., Proc. Natl. Acad. Sci. USA 83:8258-8262 (1986); U.S. Pat. No. 4,935,233; and U.S. Pat. No. 4,751,180.

The polypeptide linker may generally be from 1 to about 50 amino acids in length. ScFv joined with a polypeptide linker of at least 12 amino acids predominantly forms monomers while scFv joined with a linker of 3-11 amino acids may be sterically prohibited from folding into a monomeric form and, instead, associate with a second scFv to form a dimer. scFv joined with linkers of less than 3 amino acids may form predominantly trimers or tetramers depending upon the linker length, composition and scFv variable region orientation.

Within certain embodiments, scFv will be encoded by a polynucleotide that comprises a first polynucleotide encoding a V_{H} region and a second polynucleotide encoding a V_{L} region. Polynucleotides encoding preferred scFv further comprise a third polynucleotide that encodes a linker of at least 1 amino acid, preferable at least 3 amino acids. More preferred third polynucleotides encode linkers of between 3 and 11 amino acids. Most preferred third polynucleotides encode linkers of at least 12 amino acids.

Within still further embodiments, one or more polynucleotide encoding an affinity tag may be operably linked either 5′ and/or 3′ to a polynucleotide encoding an scFv of the present invention. As exemplified herein, suitable affinity tags include, but are not limited to, hexahistidine (i.e. His-His-His-His-His-His-), or multiples thereof, and the FLAG-tag (i.e. Asp-Tyr-Lys-Asp-Asp-Asp-Asp-Lys). When expressed in the context of an scFv polypeptide, such affinity tags may be usefully employed in detection assays, utilizing, for example, Ni or an epitope-specific antibody in the case of hexahistidine and the FLAG-tag, respectively. Affinity tags may alternatively be utilized to facilitate the direct interaction between an APC- and/or DC-specific scFv, including but not limited to an anti-DEC205 or an anti-CD11c scFv as disclosed herein, and a lipid moiety, such as a metal-chelating lipid as, for example, nitrilotriacetic acid ditetradecylamine (NTA-DTDA) as presented within PCT Patent Application Publication Nos. WO00064471 and WO09855853, each of which patent application is incorporated by reference herein in its entirety.

The ligated polynucleotide sequences may be operably linked to suitable transcriptional or translational regulatory elements to achieve expression and translation of the scFv ex vivo or in vivo. The regulatory elements responsible for expression of the scFv coding region are generally located 5′ to the polynucleotide sequence encoding the amino-terminal V_{H} or V_{L} region. Similarly, stop codons required to end translation and transcription termination signals may be present 3′ to the polynucleotide encoding the carboxy-terminal V_{H} or V_{L} region.

It will be appreciated that scFv of the present invention may be employed in methods for targeting antigens, including protein-antigens, to APC and/or DC. Suitable antigens include antigens from a wide variety of bacterial, parasitic and/or viral organisms, as indicated elsewhere herein. Accordingly, vectors expressing scFv may be engineered to accommodate the in-frame fusion of polynucleotides encoding antigens of any organism. For example, standard recombinant DNA methodology may be employed to introduce one or more cloning site immediately 3′ to the scFv coding region to facilitate the convenient, in-frame subcloning into the vector of an antigen encoding polynucleotide. The scFv/antigen fusion protein resulting from expression of the fusion construct will find utility in targeting the antigen to a surface molecule, such as a receptor molecule, on an antigen-presenting and/or dendritic cell.

Antigens Utilized in scFv/Antigen Complexes

As described above, scFv of the present invention may be employed to introduce one or more antigens into an antigen-presenting cell such as a dendritic cell. Within certain embodiments are provided complexes between scFv, that specifically bind to A PC and/or DC, and antigens such as, for example, infectious disease antigens, autoimmune disease antigens, and cancer antigens, including tissue-specific antigens and/or tumor-specific antigens.

As used herein, the term “antigen” as used in the context of scFv/antigen complexes broadly encompasses such antigens as protein-antigens, including glycoprotein-antigens, lipoprotein-antigens, and phosphoprotein-antigens. For example, it will be understood that scFv/antigen complexes, such as scFv/antigen fusion proteins, may undergo in vivo post-translational modifications wherein the protein-antigen may be glycosylated, lipidated, phosphorylated or the like. Within certain embodiments of the present invention, scFv may be complexed with one or more antigen that is encapsulated by, incorporated within, and/or associated with a lipid membrane, a lipid bi-layer, and/or a lipid complex such as, for example, a liposome, a vesicle, a micelle and/or a microsphere. Within such embodiments, therefore, the term “antigen” encompasses such liposomes, vesicles, micelles and/or microspheres that comprise an antigen, such as a protein-antigen, including glycoprotein-antigens and/or lipoprotein-antigens.

Thus, the present invention contemplates scFv/antigen complexes wherein the antigen is a protein-antigen encoded by a polynucleotide obtained from a virus, parasite or bacterium that is a causative agent of an infectious disease. Provided are protein-antigens encoded by polynucleotides from viral organisms including, but not limited to, human immunodeficiency virus (HIV), a herpes virus, and an influenza virus. Also provided are protein-antigens from parasitic organisms including, but not limited to, Leishmania (e.g., L. major and L. donovani) and from bacterial organisms including, but not limited to, Mycobacteria (e.g., M. tuberculosis and M. bovis), Chlamydia (e.g., C. trachomatis and C. pneumoniae), and Ehrlichia (e.g., E. sennetsu, E. chaffeensis, E. ewingii, and E. phagocytophila).

Exemplified herein are scFv/antigen complexes comprising scFv that specifically bind to the DC-restricted surface receptor molecules DEC-205 or CD11c, and the protein-antigen 85B from Mycobacteria tuberculosis. It has previously been shown that a DNA vaccine expressing 85B (pcDNA.85) induces protective cellular immune responses against aerosol infection with M. tuberculosis in mice. Palendira et al., Infection and Immunity 70(4):1949-1956 (2002) and U.S. Pat. No. 6,384,018. As part of the present invention, a polynucleotide encoding 85B was fused in-frame with and to the 3′-end of a polynucleotide encoding a scFv constructed from the anti-DEC-205 hybridoma NLDC-145.

Within certain aspects, the protein-antigen is an M. tuberculosis antigen selected from the group consisting of 85B, MPT64, and ESAT-6 disclosed herein in SEQ ID NO: 14, SEQ ID NO: 16, and SEQ ID NO: 18, respectively. Other aspects provide that the protein-antigen is a fragment, derivative or variant of 85B, MPT64, or ESAT-6. Preferred variants of the protein-antigens 85B, MPT64, or ESAT-6 exhibit at least about 70%, more preferably at least about 80% or 90% and most preferably at least about 95% or 98% sequence identity to the polypeptide disclosed herein in SEQ ID NO: 14, SEQ ID NO: 16, and/or SEQ ID NO: 18.

Equally suited antigens for preparing scFv/antigen complexes of the present invention include extracellular mycobacterial antigens, disclosed in U.S. Pat. No. 5,108,745 and the 79 kDa antigen of M. bovis Bacille Calmette Guerin (BCG), disclosed in U.S. Pat. Nos. 6,045,798 and 5,330,754. Other antigens that may be employed in scFv/antigen complexes included the immunostimulatory peptides presented in U.S. Pat. Nos. 6228,371, 6,214,543, 6,087,163, and 4,889,800.

Additionally, U.S. Pat. No. 6,060,259 discloses Mycobacterium protein-antigens, in particular those of M. bovis, having molecular weights between approximately 44.5 and 47.5 kDa that may be employed in scFv/antigen complexes of the present invention. U.S. Pat. No. 5,840,855 provides 540 and 517 amino acid protein-antigens, and corresponding polynucleotides, from Mycobacterium tuberculosis.

ScFv/Antigen Complexes

Within certain embodiments, the present invention provides complexes between scFv and antigens, including protein-antigens and between scFv and lipids, such as metal chelating lipids. Such complexes may be achieved by any methodology available in the art. Most commonly, scFv/antigen complexes are formed through chemical means, such as by conventional coupling techniques, or are expressed as fusion proteins encoded by polynucleotides that encode antibody heavy and light chain variable regions. Other embodiments of the present invention provide that scFv/antigen complexes may further comprise one or more lipid moiety to create scFv/lipid/antigen complexes.

For example, any of the scFv disclosed herein may be chemically coupled to an antigen using a dehydrating agent such as dicyclohexylcarbodiimide (DCCI) to form a bond, such as a peptide bond between the scFv and the antigen. Alternatively, linkages may be formed through sulfhydryl groups, epsilon amino groups, carboxyl groups or other reactive groups present in the antigens, using commercially available reagents. (Pierce Co., Rockford, Ill.).

As noted above, scFv of the present invention may also be complexed with one or more antigen that is encapsulated by, incorporated within, and/or associated with a lipid membrane, a lipid bi-layer, and/or a lipid complex such as, for example, a liposome, a vesicle, a micelle and/or a microsphere. Within such embodiments, complex formation may be achieved by cross-linking the scFv to the liposome, vesicle, micelle and/or microsphere following standard methodology that is readily available in the art. See, e.g., Metselaar et al., Mini Rev. Med. Chem. 2(4):319-29 (2002) and references cited therein. Suitable methods for preparing lipid-based antigen delivery systems that may be employed with the scFv of the present invention are described in O'Hagen et al., Expert Rev. Vaccines 2(2):269-83 (2003); O'Hagan, Curr. Durg Targets Infect. Disord. 1(3):273-86 (2001); Zho et al., Biosci Rep. 22(2):355-69 (2002); Chikh et al., Biosci Rep. 22(2):339-53 (2002); Bungener et al., Biosci. Rep. 22(2):323-38 (2002); Park, Biosci Rep. 2(2):267-81 (2002); Ulrich, Biosci. Rep. 22(2):129-50; Lofthouse, Adv. Drug Deliv. Rev. 54(6):863-70 (2002); Zhou et al., J. Immunother. 25(4):289-303 (2002); Singh et al., Pharm Res. 19(6):715-28 (2002); Wong et al., Curr. Med. Chem. 8(9):1123-36 (2001); and Zhou et al., Immunomethods 4(3):229-35 (1994).

Depending upon the precise application contemplated, scFv of the present invention may be complexed with one or more lipid and/or lipid encapsulated antigen through an affinity tag such as, for example, hexahistidine or a FLAG-tag and described herein above. According to such exemplary embodiments, a metal-chelating lipid may be employed such as, for example, nitrilotriacetic acid ditetradecylamine (NTA-DTDA) as presented within PCT Patent Application Publication Nos. WO00064471 and WO09855853, each of which patent application is incorporated by reference herein in its entirety.

Equally suited to the practice of the present invention are scFv/antigen complexes expressed as fusion proteins comprising an scFv operably linked with an antigen. scFv/antigen fusion proteins may be prepared using conventional recombinant DNA methodology wherein the 3′-end of a first polynucleotide encoding an scFv is ligated in-frame with the 5′-end of a second polynucleotide encoding one or more protein-antigen. Accordingly, the first polynucleotide and the second polynucleotide are operably linked such that they encode a fusion protein comprising the scFv and one or more protein-antigens.

More preferred embodiments provide that the first polynucleotide and the second polynucleotide are operably linked by a third polynucleotide that is ligated in-frame between the 3′-end of the first polynucleotide and the 5′-end of the second polynucleotide such that a polypeptide linker is encoded between the scFv and the protein-antigen coding regions.

Within certain embodiments, the polynucleotide encoding the scFv/antigen fusion protein is a component of a vector, such as a plasmid vector or a viral vector, for facilitating expression of the fusion protein. Preferably, the vector comprises a transcriptional promoter operably linked 5′ to the scFv encoding polynucleotide and a translational stop and/or transcription termination signal 3′ to the protein-antigen(s) coding region.

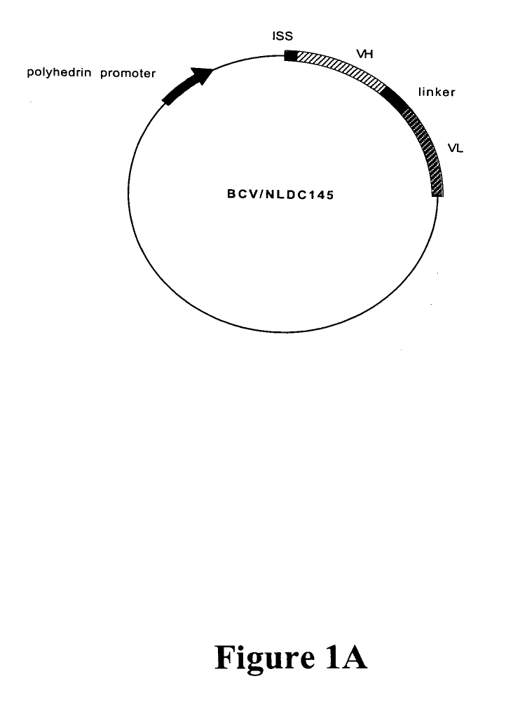

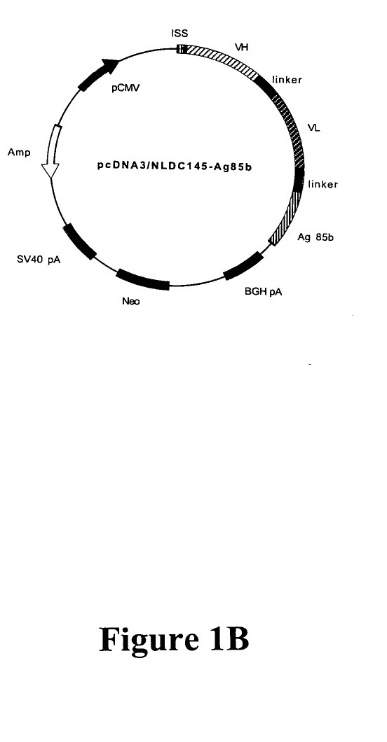

Exemplary vectors comprising a polynucleotide encoding inventive scFvs include the pBCV/NLDC-145 baculovirus expression vector described in Example 1 and the pcDNA3-NLDC-145 plasmid vector presented in FIG. 1A. An exemplary vector comprising a first polynucleotide encoding an scFv (anti-DEC-205 or anti-CD11c) and a second polynucleotide encoding the mycobacterial protein-antigen 85B is the pcDNA3-NLDC-85 and pcDNA3-N418-85 plasmid vectors presented herein in FIG. 1B and described in the Examples. The nucleotide sequence of scFv NLDC-85, scFv N418-85, and pcDNA3 are presented herein in SEQ ID NO: 8, SEQ ID NO: 3, and SEQ ID NO: 9, respectively.

Expression may be achieved in any appropriate host-cell that has been transformed or transfected with an expression vector that contains the necessary elements for transcription and translation and that contains a polynucleotide encoding an scFv or scFv/antigen of the present invention. Suitable host cells include prokaryotes, yeast and higher eukaryotic cells. Preferably, the host cells employed are bacterial (E. coli), yeast, insect, or a mammalian cell line such as COS or CHO.

In general, scFv-based complexes (whether formed by crosslinking, as fusion proteins, and/or by coupling to a lipid moiety) and polynucleotides as described herein are isolated. An “isolated” polypeptide or polynucleotide is one that is removed from its original environment. Preferably, complexes are at least about 90% pure, more preferably at least about 95% pure and most preferably at least about 99% pure.

ScFv and scFv/antigen complexes may be isolated from culture supernatants by utilizing suitable host cell/vector systems that secrete the scFv or scFv/antigen fusion proteins into culture media. For example, total protein may be concentrated using a commercially available filter and applied to a suitable purification matrix such as an affinity matrix or an ion exchange resin. One or more chromatography steps may be employed to further purify a recombinant polypeptide. In addition, scFv and/or scFv/antigen fusion proteins may further utilize a polypeptide affinity tag, such as hexahistidine (i.e. His-His-His-His-His-His-), or multimers thereof, and the FLAG tag polypeptide exemplified herein (Asp-Tyr-Lys-Asp-Asp-Asp-Asp-Lys), to facilitate detection and/or purification of the scFv or scFv/antigen fusion protein from the culture supernatant. Such affinity tags may also be employed to facilitate complex formation between scFv and/or scFv/antigen complexes and a lipid, such as a metal chelating lipid.

ScFv and scFv/Antigen Fragments Derivatives and Variants

It will be appreciated that scFv and scFv/antigen complexes according to the present invention encompass fragments, derivatives, and variants of either or both of the heavy and light chain variable regions and/or the antigen so long as the fragments, derivatives, and variants do not substantially affect the functional properties of the scFv and/or the antigen.

A polypeptide or protein “fragment, derivative, and variant,” as used herein, is a polypeptide or protein that differs from a native polypeptide or protein in one or more substitutions, deletions, additions and/or insertions, such that the functional activity of the polypeptide or protein is not substantially diminished. In other words, the ability of a variant to specifically bind to an antigen-presenting cell (APC) and/or a dendritic cell (DC) surface molecule or to be internalized and/or processed by the APC and/or DC may be enhanced or unchanged, relative to the scFv and/or antigen, or may be diminished by less than 50%, and preferably less than 20%, relative to the native protein, without affecting the efficacy of the resulting scFv and/or scFv/antigen complex.

Such fragments, derivatives, and variants may generally be identified by modifying amino acid sequence of the scFv V_{H} and/or V_{L} moiety and evaluating the reactivity of the modified scFv with APC and/or DC or with antisera raised against the native protein-antigen. Such modification and evaluation may be achieved through routine application of molecular and cell biology techniques that are well known in the art.

Polypeptide fragments, derivatives, and variants preferably exhibit at least about 70%, more preferably at least about 80% or 90% and most preferably at least about 95% or 98% sequence identity to the native polypeptide or protein. Preferably, variants contain “conservative amino acid substitutions” as defined as a substitution in which one amino acid is substituted for another amino acid that has similar properties, such that the secondary structure and hydropathic nature of the polypeptide is substantially unchanged. Amino acid substitutions may generally be made on the basis of similarity in polarity, charge, solubility, hydrophobicity, hydrophilicity and/or the amphipathic nature of the residues. For example, negatively charged amino acids include aspartic acid and glutamic acid; positively charged amino acids include lysine and arginine; and amino acids with uncharged polar head groups having similar hydrophilicity values include leucine, isoleucine and valine; glycine and alanine; asparagine and glutamine; and serine, threonine, phenylalanine and tyrosine. Other groups of amino acids that may represent conservative changes include: (1) ala, pro, gly, glu, asp, gln, asn, ser, thr; (2) cys, ser, tyr, thr; (3) val, ile, leu, met, ala, phe; (4) lys, arg, his; and (5) phe, tyr, trp, his. A variant may also, or alternatively, contain nonconservative changes.

Variants may additionally, or alternatively, be modified by, for example, the deletion or addition of amino acids that have minimal influence on the surface molecule specific binding, secondary structure and hydropathic nature of the scFv and/or protein-antigen.

Functional fragments, derivatives, and variants of a polypeptide may be identified by first preparing fragments of the polypeptide by either chemical or enzymatic digestion of the polypeptide, or by mutation analysis of the polynucleotide that encodes the polypeptide and subsequent expression of the resulting mutant polypeptides. The polypeptide fragments or mutant polypeptides are then tested to determine which portions retain biological activity, using, for example, the representative assays provided below.

Fragments, derivatives, and variants of the inventive polypeptides may also be generated by synthetic or recombinant means. Synthetic polypeptides having fewer than about 100 amino acids, and generally fewer than about 50 amino acids, may be generated using techniques well known to those of ordinary skill in the art. For example, such polypeptides may be synthesized using any of the commercially available solid-phase techniques, such as the Merrifield solid-phase synthesis method, where amino acids are sequentially added to a growing amino acid chain. Merrifield, J. Am. Chem. Soc. 85:2149-2154 (1963). Equipment for automated synthesis of polypeptides is commercially available from suppliers such as Perkin Elmer/Applied BioSystems, Inc. (Foster City, Calif.), and may be operated according to the manufacturer's instructions. Variants of a native polypeptide may be prepared using standard mutagenesis techniques, such as oligonucleotide-directed, site-specific mutagenesis. Kunkel, Proc. Natl. Acad. Sci. USA 82:488-492 (1985). Sections of polynucleotide sequence may also be removed using standard techniques to permit preparation of truncated polypeptides.

As used herein, the term “variant” comprehends nucleotide or amino acid sequences different from the specifically identified sequences, wherein one or more nucleotides or amino acid residues is deleted, substituted, or added. Variants may be naturally occurring allelic variants, or non-naturally occurring variants. Variant sequences (polynucleotide or polypeptide) preferably exhibit at least 70%, more preferably at least 80% or at least 90%, more preferably yet at least 95%, and most preferably, at least 98% identity to a sequence of the present invention. The percentage identity is determined by aligning the two sequences to be compared as described below, determining the number of identical residues in the aligned portion, dividing that number by the total number of residues in the inventive (queried) sequence, and multiplying the result by 100. In addition to exhibiting the recited level of sequence similarity, variant sequences of the present invention preferably exhibit a functionality that is substantially similar to the functionality of the sequence against which the variant is compared.

Polynucleotide sequences may be aligned, and percentages of identical nucleotides in a specified region may be determined against another polynucleotide, using computer algorithms that are publicly available. Two exemplary algorithms for aligning and identifying the similarity of polynucleotide sequences are the BLASTN and FASTA algorithms. The alignment and identity of polypeptide sequences may be examined using the BLASTP algorithm. BLASTX and FASTX algorithms compare nucleotide query sequences translated in all reading frames against polypeptide sequences. The FASTA and FASTX algorithms are described in Pearson and Lipman, Proc. Natl. Acad. Sci. USA 85:2444-2448 (1988); and in Pearson, Methods in Enzymol. 183:63-98 (1990). The FASTA software package is available from the University of Virginia by contacting David Hudson, Assistant Provost for Research, University of Virginia, P.O. Box 9025, Charlottesville, Va. 22906-9025. The FASTA algorithm, set to the default parameters described in the documentation and distributed with the algorithm, may be used in the determination of polynucleotide variants. The readme files for FASTA and FASTX Version 2.0× that are distributed with the algorithms describe the use of the algorithms and describe the default parameters.

The BLASTN software is available on the NCBI anonymous FTP server and is available from the National Center for Biotechnology Information (NCBI), National Library of Medicine, Building 38A, Room 8N805, Bethesda, Md. 20894. The BLASTN algorithm Version 2.0.6 [Sep. 10, 1998] and Version 2.0.11 [Jan. 20, 2000] set to the default parameters described in the documentation and distributed with the algorithm, is preferred for use in the determination of variants according to the present invention. The use of the BLAST family of algorithms, including BLASTN, is described at NCBI's website and in the publication of Altschul et al., “Gapped BLAST and PSI-BLAST: a new generation of protein database search programs,” Nucleic Acids Res. 25:3389-3402 (1997).

The following running parameters are preferred for determination of alignments and identities using BLASTN that contribute to the E values and percentage identity for polynucleotides: Unix running command with the following default parameters: blastall -p blastn -d embldb -e 10 -G 0 -E 0 -r 1 -v 30 -b 30 -i queryseq -o results; and parameters are: -p Program Name [String]; -d Database [String]; -e Expectation value (E) [Real]; -G Cost to open a gap (zero invokes default behavior) [Integer]; -E Cost to extend a gap (zero invokes default behavior) [Integer]; -r Reward for a nucleotide match (BLASTN only) [Integer]; -v Number of one-line descriptions (V) [Integer]; -b Number of a lignments to show (B) [Integer]; -i Query File [File In]; -o BLAST report Output File [File Out] Optional.

The “hits” to one or more database sequences by a queried sequence produced by BLASTN, BLASTP, FASTA, or a similar algorithm, align and identify similar portions of sequences. The hits are arranged in order of the degree of similarity and the length of sequence overlap. Hits to a database sequence generally represent an overlap over only a fraction of the sequence length of the queried sequence.

The percentage identity of a polynucleotide or polypeptide sequence is determined by aligning polynucleotide and polypeptide sequences using appropriate algorithms, such as LASTN or BLASTP, respectively, set to default parameters; identifying the number of identical nucleic or amino acids over the aligned portions; dividing the number of identical nucleic or amino acids by the total number of nucleic or amino acids of the polynucleotide or polypeptide of the present invention; and then multiplying by 100 to determine the percentage identity. By way of example, a queried polynucleotide having 220 nucleic acids has a hit to a polynucleotide sequence in the EMBL database having 520 nucleic acids over a stretch of 23 nucleotides in the alignment produced by the BLASTN algorithm using the default parameters. The 23-nucleotide hit includes 21 identical nucleotides, one gap and one different nucleotide. The percentage identity of the queried polynucleotide to the hit in the EMBL database is thus 21/220 times 100, or 9.5%. The identity of polypeptide sequences may be determined in a similar fashion.

The BLASTN and BLASTX algorithms also produce “Expect” values for polynucleotide and polypeptide a lignments. The Expect value (E) indicates the number of hits one can “expect” to see over a certain number of contiguous sequences by chance when searching a database of a certain size. The Expect value is used as a significance threshold for determining whether the hit to a database indicates true similarity. For example, an E value of 0.1 assigned to a polynucleotide hit is interpreted as meaning that in a database of the size of the EMBL database, one might expect to see 0.1 matches over the aligned portion of the sequence with a similar score simply by chance. By this criterion, the aligned and matched portions of the sequences then have a probability of 90% of being related. For sequences having an E value of 0.01 or less over aligned and matched portions, the probability of finding a match by chance in the EMBL database is 1% or less using the BLASTN algorithm. E values for polypeptide sequences may be determined in a similar fashion using various polypeptide databases, such as the SwissProt database.

According to one embodiment, “variant” polynucleotides and polypeptides, with reference to each of the polynucleotides and polypeptides of the present invention, preferably comprise sequences having the same number or fewer nucleic or amino acids than each of the polynucleotides or polypeptides of the present invention and producing an E value of 0.01 or less when compared to the polynucleotide or polypeptide of the present invention. That is, a variant polynucleotide or polypeptide is any sequence that has at least a 99% probability of being related as the polynucleotide or polypeptide of the present invention, measured as having an E value of 0.01 or less using the BLASTN or BLASTX algorithms set at the default parameters. According to a preferred embodiment, a variant polynucleotide is a sequence having the same number or fewer nucleic acids than a polynucleotide of the present invention that has at least a 9 9% probability of being related as the polynucleotide of the present invention, measured as having an E value of 0.01 or less using the BLASTN algorithm set at the default parameters. Similarly, according to a preferred embodiment, a variant polypeptide is a sequence having the same number or fewer amino acids than a polypeptide of the present invention that has at least a 99% probability of being related as the polypeptide of the present invention, measured as having an E value of 0.01 or less using the BLASTP algorithm set at the default parameters.

In addition to having a specified percentage identity to an inventive polynucleotide or polypeptide sequence, variant polynucleotides and polypeptides preferably have additional structure and/or functional features in common with the inventive polynucleotide or polypeptide. Polypeptides having a specified degree of identity to a polypeptide of the present invention share a high degree of similarity in their primary structure and have substantially similar functional properties. In addition to sharing a high degree of similarity in their primary structure to polynucleotides of the present invention, polynucleotides having a specified degree of identity to, or capable of hybridizing to, an inventive polynucleotide preferably have at least one of the following features: (i) they contain an open reading frame or partial open reading frame encoding a polypeptide having substantially the same functional properties as the polypeptide encoded by the inventive polynucleotide; or (ii) they contain identifiable domains in common.

Suitable variants of the scFv NLDC-145-85B and scFv N418-85B disclosed herein comprise sequence variations within the amino acid sequences of the scFv and/or 85B moieties. For example, the present invention contemplates protein conjugates wherein the scFv NLDC-145-85B and scFv N418-85B are at least 70% identical with the amino acid sequences encoded by the polynucleotides recited in SEQ ID NOs: 8 and 3, respectively. More preferred are scFv NLDC-145-85B and scFv N418-85B that are at least 80%, 90%, 95% and 98% identical to the amino acid sequences recited in SEQ ID NOs: 8 and 3, respectively.

Methods for Use

ScFv-based complexes of the present invention, including compositions thereof, will find utility in a number of methods as exemplified by those disclosed herein.

Within certain embodiments, the present invention provides, for example, ex vivo methods for introducing an antigen into an antigen-presenting cell (APC) and/or a dendritic cell (DC). By these methods, APC and/or DC are isolated from a patient sample and contacted with the isolated APC and/or DC with an scFv/antigen complex under conditions and for such a time as required to permit the antigen to enter the APC and/or DC.

Each of the ex vivo methods disclosed herein requires the isolation of antigen-presenting cells (APC) and/or dentritic cells (DCs) from a patient sample, most preferably a human. APCs may generally be isolated from any of a variety of biological fluids and organs, including tumor and peritumoral tissues, and may be autologous, allogeneic, syngeneic or xenogeneic cells. Dendritic cells and progenitors may be obtained from peripheral blood, bone marrow, tumor-infiltrating cells, peritumoral tissues-infiltrating cells, lymph nodes, spleen, skin, umbilical cord blood or any other suitable tissue or fluid. DCs may be differentiated ex vivo by adding a combination of cytokines such as GM-CSF, IL-4, IL-13 and/or TNFα to cultures of monocytes harvested from peripheral blood. Alternatively, CD34 positive cells harvested from peripheral blood, umbilical cord blood or bone marrow may be differentiated into dendritic cells by adding to the culture medium combinations of GM-CSF, IL-3, TNFα, CD40 ligand, LPS, flt3 ligand and/or other compound(s) that induce differentiation, maturation and proliferation of dendritic cells.

Dendritic cells are conveniently categorized as “immature” and “mature” cells, which allow a simple way to discriminate between two well characterized phenotypes. Immature dendritic cells are characterized as APC with a high capacity for antigen uptake and processing, which correlates with the high expression of Fcγ receptor and mannose receptor (MR). The mature phenotype is typically characterized by a lower expression of these markers, but a high expression of cell surface molecules responsible for T-cell activation such as class I and class II MHC, adhesion molecules (e.g., CD54 and CD11) and costimulatory molecules (e.g., CD40, CD80, CD86 and 4-1 BB).

As noted above, certain preferred embodiments of the present invention employ dendritic cells (DCs), or progenitors thereof, as antigen-presenting cells. Dendritic cells express a number of surface molecules, exemplified herein by DEC-205 and CD11c, which are restricted in expression to DC. It is contemplated, however, that DC may, alternatively, be engineered to express specific cell-surface receptors or ligands that are not commonly found on dendritic cells in vivo or ex vivo, and such modified dendritic cells are within the scope of the present invention.

Alternative embodiments of the present invention provide in vivo methods for introducing a protein-antigen into an APC and/or a DC of a patient, preferably a human patient. Such methods comprise the step of administering to the patient a composition comprising an scFv/antigen complex as disclosed herein above and as exemplified below by the scFv NLDC-85B antigen complex. In related embodiments, one or more polynucleotide encoding an APC and/or DC-specific scFv/antigen may be administered thus utilizing in vivo expression of the scFv/antigen coding region.

Still further aspects of the present invention provide methods for enhancing, stimulating, suppressing, and/or blocking an immune response in a patient as well as methods for treating a disease in a patient, the methods comprising the steps of: (a) obtaining from the patient a sample comprising an antigen-presenting cell (APC) and/or a dendritic cell (DC); (b) contacting the sample with an scFv/antigen complex under conditions and for such a time as required to allow binding of the scFv/antigen complex to the APC and/or DC; and (c) administering the scFv/antigen APC and/or DC-bound complex to the patient.

Within such methods, the immune response may be a cellular response, such as a T-cell response, or an antibody response. Exemplary cellular responses include a ThI response, a Tₕ2 response, and a Cytotoxic T-cell (CTL) response. Exemplary antibody responses include IgM, IgD, IgG₃, IgG₁, IgG_{2b}, IgG₂ₐ, IgE, and IgA responses.

Within such methods, the disease may be selected from the group consisting of an infectious disease and cancer. More preferred methods provide that the infectious disease is caused by a virus, a parasite, or a bacterium. Exemplary viral organisms include, but are not limited to, human immunodeficiency virus (HIV), a herpes virus, and an influenza virus. Exemplary parasitic organisms include, but are not limited to, Leishmania (e.g., L. major and L. donovani). Exemplary bacterial organisms include, but are not limited to, Mycobacteria (e.g., M. tuberculosis and M. bovis), Chlamydia (e.g., C. trachomatis and C. pneumoniae), and Ehrlichia (e.g., E. sennetsu, E. chaffeensis, E. ewingii, and E. phagocytophila). Cancers that may be amenable to treatment with the methods of the present invention include, but are not limited to, soft tissue sarcomas, lymphomas, and cancers of the brain, esophagus, uterine cervix, bone, lung, endometrium, bladder, breast, larynx, colon/rectum, stomach, ovary, pancreas, adrenal gland and prostate.

Other aspects provide methods for inhibiting, reducing, suppressing and/or blocking the activity of a target antigen on the surface of an antigen-presenting cell (APC) and/or a dendritic cell (DC), the methods comprising the steps of: (a) obtaining a sample comprising and APC and/or a DC; (b) contacting the APC and/or DC with an scFv capable of specifically binding to the target antigen the surface of the APC and/or DC under conditions and for such a time as required to permit binding of the scFv to the APC and/or DC, wherein binding of the scFv to the APC and/or DC blocks the activity of the target antigen.

By any of the methods disclosed herein, the scFv may bind to a molecule, including a carbohydrate molecule or a protein molecule, on the surface of the APC and/or DC. Preferred surface protein molecules include, but are not limited to, the mannose receptor (MR), chemokine receptor 1 (CCR1), B7-1, B7-2, CD40, CD11c, DEC-205, a Toll-like receptor (TLR), and the Fcγ receptor (FcγR).

Within certain methods, the scFv may be complexed to an antigen wherein scFv/antigen complexes are achieved by chemical crosslinking or wherein scFv/antigen complexes are scFv/antigen fusion proteins. Alternatively, the scFv may be complexed with a liposome, a vesicle, a micelle and/or a microsphere.

The scFv and scFv/antigen complexes of the invention may be administered prophylactically or therapeutically to an individual already suffering from the disease. In either case, the efficacy of the scFv and/or scFv/antigen complex will depend upon the modulation of the patient's immune response. For example, scFv administered alone may be effective in blocking the target molecule on the APC and/or DC and, consequently, may reduce the intensity of an immune response. On the other hand, scFv/antigen complexes may stimulate an antigen-specific immune response, for example, by activating cytokine release from helper T-cells and/or by stimulating cytotoxic T-cells (CTL). In addition, or alternatively, scFv/antigen complexes may also stimulate B-cells to produce antibody including IgM, IgD, IgG₃, IgG₁, IgG_{2b}, IgG₂ₐ, IgE, and/or IgA.

ScFv- and/or scFv/antigen-based compositions may be administered to a patient in an amount sufficient to modulate the immune response. An amount adequate to accomplish this is defined as “therapeutically effective dose” or “immunogenically effective dose.” Amounts effective for this use will depend, for example, on the precise composition composition, the manner of administration, the stage and severity of the disease being treated, the weight and general state of health of the patient, and the judgment of the prescribing physician, but generally range for the initial immunization dose (that is for therapeutic or prophylactic administration) from about 0.01 mg to about 50 mg per 70 kilogram patient, more commonly from about 0.5-1 mg to about 10-15 mg per 70 kg of body weight. Boosting dosages are typically from about 0.01 mg to about 50 mg of peptide, more commonly about 0.5-1 mg to about 10-15 mg, using a boosting regimen over weeks to months depending upon the patient's response and condition. A suitable protocol would include injection at time 0, 2, 6, 8, 10 and 14 weeks, followed by booster injections at 24 and 28 weeks. Booster injections can be from one, two, three, four, five or more. Initial and booster injection amounts and timing are determined based on the judgment of the physician and the antigen being administered. In one embodiment, the initial and booster dose is 1.3 mg, 4 mg, or 13 mg, administered via intramuscular injection, with at least one and up to 3 booster injections at 8 week intervals, or at least one and up to 4 booster injections at 6 week intervals.

Within specific methods for stimulating an immune response against a mycobacterial antigen, a prime/boost regimen may be employed wherein a first immunization comprises pcDNA3/scFv/NLDC-85B and pcDNA3/scFv/N418-85B vectors and/or protein followed by a second immunization with M. bovis Bacille Calmette Guerin (BCG).

It has been shown that bacterial infection of dendritic cells, in particular mycobacterial infection, results in the upregulation of the regulatory cytokine IL-12 and of inflammatory cytokines such as IL-1, IL-6, and tumor necrosis factor (TNF) which may contribute to the acquired specific resistance against bacterial infection and may promote the development of delayed-type hypersensitivity (DTH). Demangel et al., Immunol. and Cell Biol. 78:318-324 (2000). Accordingly, depending on the particular application contemplated, it may be desirable to employ one or more of these, or other, cytokines in conjunction with the scFv/antigen to improve therapeutic efficacy over that achieved with scFv/antigens alone. Thus, compositions and methods of the present invention may further comprise a cytokine selected from the group consisting of IL-1, IL-4, IL-6, IL-12, IFNγ, GM-CSF, and TNF. Alternatively or additionally, compositions of the present invention may comprise a lipopolysaccharide (LPS) or other modulator of the DC response to antigen. Treatment regimens may employ one or more cytokine administered separately from administration of the scFv/antigen complex.

The following Examples are offered by way of illustration not limitation.

### EXAMPLE 1

### Generation of Plasmid Constructs Encoding Anti-DEC-205 and Anti-CD11c Single-Chain Variable Region Fragments (scFvs)

This example discloses the generation of scFv that specifically bind to DEC-205 and CD11c.

Hybridoma cell lines expressing rat anti-DEC-205 monoclonal antibody NLDC-145 (ATCC Accession No. HB-290; Inaba et al., Cellular Immunology 163:148-56 (1995) and Witmer-Pack et al., Cellular Immunology 163:157-62 (1995)) and the hamster anti-CD11c monoclonal antibody hybridoma N418 (ATCC Accession No. HB-224; Metlay et al., i J. Exp. Med. 171:1753-1771 (1990)), were cultured in RPMI 1640 supplemented with 5% fetal bovine serum (FBS), 50 μM β-mercaptoethanol (BME) and 2 mM Glutamine (Gln). Monoclonal antibodies were purified from culture supernatants by chromatography on a protein-G column (Pharmacia; Peapack, N.J., USA). Sf21 insect cells (Clontech; Palo Alto, Calif., USA) were propagated at 27° C. in Grace's Medium supplemented with 10% FBS and 2 mM Gln (Gibco BRL/Life Technologies). Passages 1 and 2 were performed on Sf21 monolayers, and passage 3 in suspension culture with culture medium supplemented with 1% Pluronic F-68® (BASF Corporation).

Total mRNA was extracted from NLDC-145 and N418 hybridoma cells using RNAzol (Cinna/Tel-Test, Inc.; Friendswood, Tex., USA), and first strand complementary DNA (cDNA) synthesized using reverse transcriptase and random hexamers by employing the Recombinant Phage Antibody system (Pharmacia; Uppsala, Sweden). Heavy and light chain variable regions (V_{H} and V_{L}) of the NLDC-145 and N418 rat immunoglobulin genes were PCR amplified from these cDNAs using a collection of primers originally designed for murine antibodies (Recombinant Phage Antibody System, Pharmacia). The nucleotide and amino acid sequences of scFv NLDC145 are presented herein in SEQ ID NOs: 4 and 7, respectively. The nucleotide and amino acid sequences of scFv N418 are presented herein in SEQ ID NOs: 1 and 2, respectively.

Polynucleotides encoding the V_{H} and V_{L} regions were operably linked to a polynucleotide encoding a peptide linker having the amino acid sequence Gly-Gly-Gly-Gly-Ser-Gly-Gly-Gly-Gly-Ser-Gly-Gly-Gly-Gly-Ser such that the V_{H} coding region was ligated 5′ to the linker encoding polynucleotide and the V_{L} coding region was ligated 3′ to the linker encoding polynucleotide to yield a 750 bp long polynucleotide fragment encoding an NLDC-145-derived anti-DEC-205 scFv having the arrangement V_{H}-linker-V_{L}.

For immunogenicity experiments, the scFvs were fused to the gene encoding the mycobacterial antigen Ag85B. Kamath et al., Clinical and Experimental Immunology 120:476-482 (2000). To allow expression of the product, the N-terminal end of the scFv was fused to HBM sequence. Reavy et al., Protein Expression and Purification 18:221-228 (2000). Moreover, in order to facilitate its detection and purification, the C-terminal end of the scFv was fused to the DYKDDDDK peptide (FLAG) and the resulting product subcloned in pCDNA3 expression vector (Invitrogen).

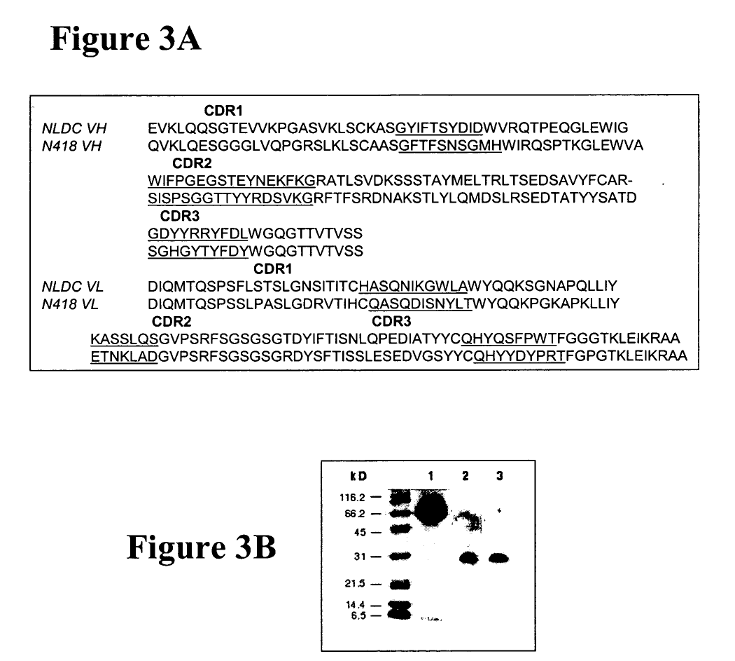

The genes encoding the variable regions of the heavy (V_{H}) and light (V_{L}) chains of NLDC-145 and N418 were amplified by PCR. Deduced amino acid sequence of the resulting products displayed the typical architecture of immunoglobulin variable domains (FIG. 3A). Each V_{H} fragment was then bound to its V_{L} partner by use of a spacer encoding a 15 amino-acid flexible linker, yielding scFv constructs ScNLDC and ScN418.

### EXAMPLE 2

### Expression and Purification of Anti-DEC-205 scFv NLDC-145 and Anti-CD11c scFv N418

This example discloses the expression and purification of the anti-DEC-205 scFv designated scFv NLDC-145 and the anti-CD11c scFv designated scFv N418.

To examine the binding specificities of ScNLDC and ScN418, these NLDC-145-derived anti-DEC-205 scFv and N418-derived anti-CD11c scFv were expressed in a baculovirus expression system. For this purpose, the N-terminus of ScNLDC and ScN418 were fused to the honeybee melittin leader sequence (HBM; Reavy et al., Protein Expression and Purification 18:221-228 (2000)), and their C-terminal end to a FLAG peptide and a hexahistidine tail. FIG. 3B shows that cells infected with ScNLDC recombinant baculovirus released a 30 kD protein, identified as ScNLDC by Western blot analysis using anti-FLAG and anti-hexahistidine antibodies. Comparably to ScNLDC, ScN418 was successfully produced and secreted by insect cells infected with ScN418 recombinant baculovirus (not shown), allowing us to purify both scFv products from insect cell culture supernatants by FLAG affinity-based chromatography.

The polynucleotide encoding the NLDC-145-derived anti-DEC-205 scFv described in Example 1 was cloned into the baculovirus expression vector pBACPak8 (Clontech; Palo Alto, Calif.) to generate the plasmid vector designated pBCV/NLDC-145 presented herein as FIG. 1A. Similarly, the polynucleotide encoding the N418-derived anti-CD11c (SEQ ID NO: 3) scFv was cloned into pBACPak8 to generate the plasmid vector designated pBCV/N418.

For expression in insect cells, the NLDC-145 scFv and the N418 scFv polynucleotides were each ligated in-frame with a polynucleotide encoding a FLAG peptide (DYKDDDDK) and a poly-Histidine tail within the transfer vector pBacPAK8 (Clontech: Palo Alto, Calif., USA). Plasmid and baculovirus DNA were co-transfected into Sf21 cells in the presence of Lipofectin (Gibco BRL/Life Technologies) and recombinant viruses amplified according to the protocol recommended by the manufacturer. The HBM-leaded recombinant protein was secreted by the virus particles and recovered from infected Sf21 culture medium. The NLDC-145 scFv and the N418 scFv polypeptides were adsorbed onto an anti-FLAG M2 affinity column as described by the manufacturer (Sigma-AldrichCorp.; St. Louis, Mo., USA) and eluted with 0.1 M glycine, with immediate neutralization by Tris 0.1 M pH 8.0. Purified NLDC-145 scFv and N418 scFv were analyzed by SDS-PAGE on a 12% acrylamide gel followed by silver staining, and by Western-blot using the anti-FLAG M2 and the anti-polyhistidine His1 antibodies (both from Sigma).

For expression in insect cells, scFv constructs tagged with a FLAG peptide and a hexahistidine tail were subcloned in the transfer vector pBacPAK8 (Clontech). Plasmid and baculovirus DNA were co-transfected into Sf21 cells in the presence of Lipofectin (Gibco BRL/Life Technologies), and recombinant viruses amplified according to the manufacturer's protocol. The HBM-leaded recombinant protein was secreted by the virus particles and could be recovered from infected Sf21 culture medium. Passage 3 supernatant was purified by affinity chromatography on an anti-FLAG M2 gel, as described by the manufacturer (Sigma). Acid elution was performed with 0.1M glycine, with immediate neutralization by Tris 0.1M pH 8.0. Elution fractions were analyzed by SDS-PAGE on a 12% acrylamide gel followed by silver staining, and by Western-blot using the anti-FLAG M2 and the anti-polyhistidine His1 antibodies (both from Sigma).

### EXAMPLE 3

### Binding of the Anti-DEC-205 scFv NLDC-145 to Langerhans Cells

This example demonstrates that scFv NLDC-145 is capable of specifically binding to murine dendritic cells (Langerhans cells).

Epidermal sheets of mouse ears were prepared as described. Halliday et al., Immunology 77(1):13-8 (1992). Epidermis were incubated with scNLDC (20 μg/ml), purified as described in Example 2, for 72 h at 4° C., followed by 10 μg/ml M2 or a 1:100 dilution of His1 for 16 h at 4° C., biotinylated goat anti-mouse antibody (1:200) 16 h at 4° C., and streptavidin-conjugated alkaline phosphatase (1:200) for 2 h at RT. Control epidermis were stained with equivalent binding site molar concentration (50 μg/ml) of purified parental NLDC-145 monoclonal antibody, followed by biotinylated goat anti-rat antibody (1:200) for 16 h at 4° C., and streptavidin-conjugated alkaline phosphatase (1:200) for 2 h at RT. ScN418 binding to CD11c was assessed on FSDCs, by incubation of fixed cells with purified ScN418 (10 μg/ml) for 1 h at RT, followed by 10 μg/ml M2 or a 1:100 dilution of His1 for for 1 h at RT, horseradish peroxidase-conjugated anti mouse antibody for 1 h at RT.

Control cells were stained with equivalent binding site molar concentration (25μg/ml) of biotinylated N418 antibody, followed by streptavidin-conjugated horseradish peroxidase for 1 h at RT. Controls included epidermal sheets incubated with secondary reagents only. Each incubation step was performed in DMEM+10% FCS and was followed by three 2 h washes in PBS with gentle agitation. After final washing, epidermis were stained with a fuchsin-based alkaline phosphatase substrate for 20 min, and mounted on glass microscope slides in Histomount. Epidermal sheets of mouse ears stained with NLDC-145 parental monoclonal antibody or with purified scFv NLDC-145, as detected via the C-terminal poly-His tail or FLAG peptide, were compared. The scFv NLDC-145 bound to the subcutaneous dendritic cells with the same specificity as did the parental monoclonal antibody.

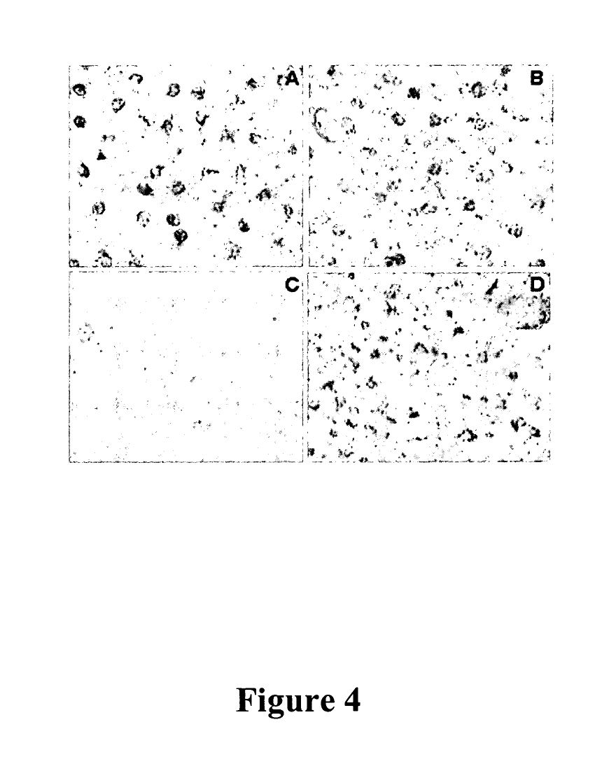

As mouse epidermal DC (or Langerhans cells) display a distinctive DEC-205^{high} phenotype (Anjuere et al., Blood 93:590-8 (1999)), we use depidermal sheets of mouse ears to examine the ability of ScNLDC to bind its target receptor. Staining of mouse epidermis with 50 μg/ml purified NLDC-145 antibody revealed the characteristic network formed by Langerhans cells (FIG. 4A). When mouse epidermal tissues were incubated with 20 μg/ml ScNLDC (an antigen binding site concentration equivalent to 50 μg/ml NLDC-145), an equivalent phenotype was detected, using either anti-hexahistidine or anti-FLAG detection antibodies (FIGS. 4B and 4D). No signal was observed in the absence of ScNLDC (FIG. 4C), demonstrating that ScNLDC retains the specificity of the parental antibody.

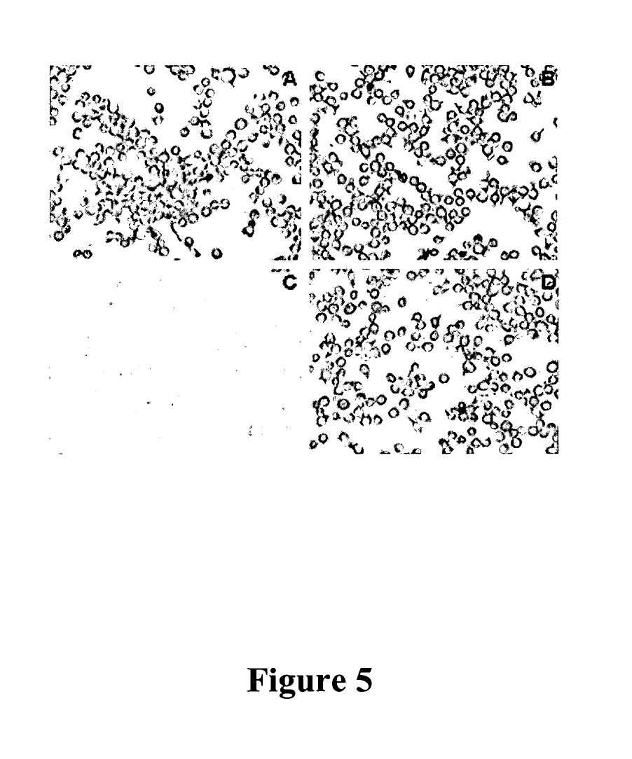

The ability of ScN418 to bind CD11c was similarly investigated on the dendritic cell line FSDC. Girolomoni et al., European J. Immunol. 25:2163-2169 (1995). Staining of FSDCs was equivalent using either 10 μg/ml N418 monoclonal antibody or an equivalent concentration of ScN418 (FIGS. 5A, 5B, and 5D). In contrast, no staining was detected in the absence of the scFv (FIG. 5C), or when the cells were incubated with N418 prior to ScN418 (not shown). Therefore, ScN418 binds CD11c with the same specificity as N418.

### EXAMPLE 4

### Generation of a Plasmid Construct Encoding an Anti-DEC-205 Single-Chain Fv- M. Tuberculosis Antigen 85B Fusion Protein

This example discloses the generation of a scFv/antigen complex comprising scFv NLDC-145, described above, fused to the protein-antigen 85B from mycobacterium tuberculosis.

A polynucleotide encoding scFv NLDC-145 was ligated in-frame with a polynucleotide encoding the mycobacterial protein-antigen 85B. Kamath et al., Infection and Immunity 67(4):1702-1707 (1999). To facilitate expression of scFv/antigen complex, a polynucleotide encoding the honeybee melittin signal peptide (HBM) was ligated 5′ to and in-frame with the polynucleotide encoding scFv NLDC-145. Tessier et al., Gene 98(2):177-83 (1991). A polynucleotide encoding a linker polypeptide (SEQ ID NO: 11) was ligated 3′ to and in-frame with the polynucleotide encoding scFv NLDC-145 and a polynucleotide encoding the M. tuberculosis protein-antigen 85B (SEQ ID NO: 13) was ligated 3′ to and in-frame with the polynucleotide encoding the linker polypeptide. This fusion polynucleotide construct was cloned into the pcDNA3 plasmid vector (Invitrogen, Carlsbad, Calif.) to generate pcDNA3-NLDC-85B. (FIG. 1B). The nucleotide sequence of scFvNLDC-145-85B is presented in SEQ ID NO: 8.

COS cells were transfected with pcDNA3-NLDC-85B and a control plasmid, pcDNA3-85B, that expresses the 85B antigen alone. Expression of the anti-DEC-205 scFv NLDC-85B fusion protein and 85B protein-antigen were readily detected in extracts of the COS cells by standard immunoblotting methodology.

### EXAMPLE 5

### Targetong of scFv NLDC-85B to Dendritic Cells Stimulates 85B-Specific T-Cell Responses

This Example demonstrates that scFv NLDC-85B specifically binds dendritic cells and facilitates a T-cell response against the M. tuberculosis 85B protein-antigen.

The immune response facilitated by plasmid vectors pcDNA3-NLDC-85B and pcDNA3-85B were compared in C57BL/6 mice. C57B1/6 female mice were supplied as specific-pathogen-free mice by the Animal Resource Centre (Perth, Australia) and were maintained under specific-pathogen-free conditions. Mice were immunized at 8 weeks of age. For intramuscular injections, 50 μg of plasmid was injected into the tibialis anterior muscle of each hindleg. For intradermal injections, the same quantity of DNA was delivered in the dermis of each ear. Control mice were immunized with pcDNA3, or with the pcDNA3 vector expressing Ag85B in the absence of scFv NLDC-145. Mice were immunized either one or two times at 2-week intervals, and sacrified 4 weeks after the last injection for immunogenicity studies.

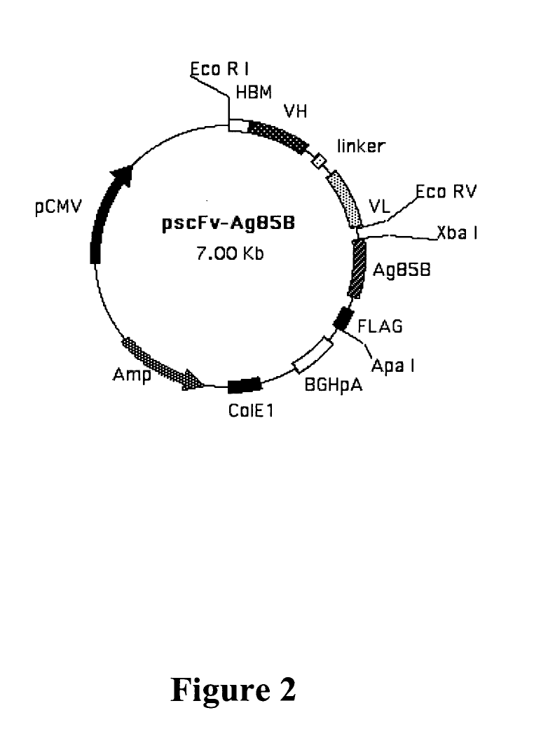

To test the impact of antigen targeting on dendritic cells, DNA vaccine vectors encoding fusion proteins between scFvs and a model mycobacterial antigen (Ag85B) were designed. Kamath et al., supra. For product detection purposes, the plasmid vectors (pScNLDC-Ag85B and pScN418-Ag85B) contained a FLAG sequence linked to the 3′ of the Ag85B gene (FIG. 2). Western blot analysis using anti-Ag85B and anti-FLAG detection antibodies identified a product expressed by COS7 cells transfected with pScNLDC-Ag85B or pScN418-Ag85B. Introduction of the targeting sequence had no impact on Ag85B expression, as COS7 cells transfected with pScNLDC-Ag85B, pScN418-Ag85B or with a plasmid encoding the Ag85B gene without scFv (pAg85B) expressed comparable amounts of Ag85B (not shown). These DNA vectors were then used to immunize mice via the intramuscular route, and compared for their ability to generate antibodies and IFN-γ secreting T cells specific of Ag85B.

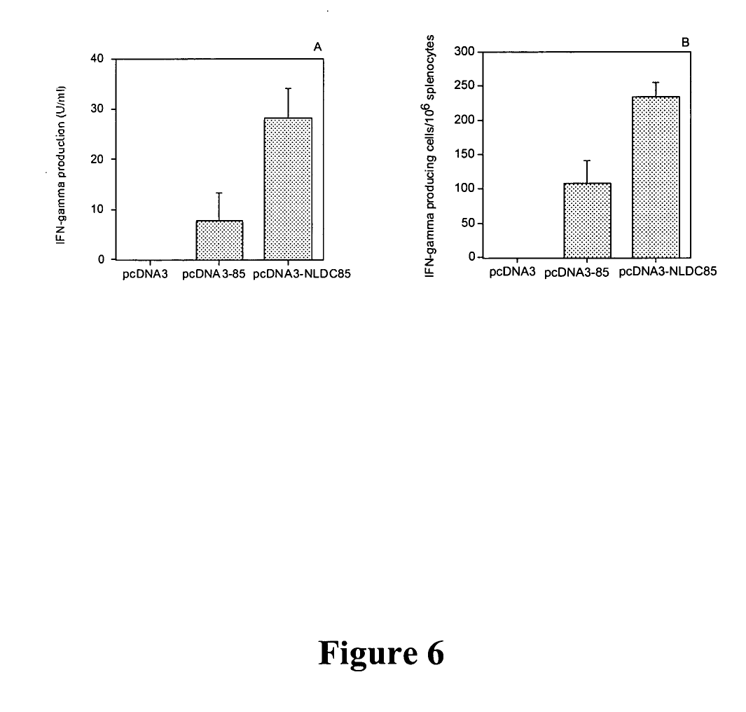

Spleens from the sacrificed mice were harvested, splenocytes isolated and cultured in the presence of M. tuberculosis antigen 85 to measure Interferon-γ (IFN-γ) release and to quantify the number of IFN-γ-secreting T-cells. Mice immunized with pcDNA3-NLDC-85 induced a stronger IFN-γ secreting T-cell response against antigen 85B than mice immunized with antigen 85B alone, with a more than two-fold increase the number of specific IFN-γ secreting T-cells. (FIG. 6). The generation of Th1-type T cells specific of Ag85B was also enhanced, as evidenced by the increased frequency of antigen specific IFN-γ secreting cells in mice immunized by one injection of pScNLDC-Ag85B.

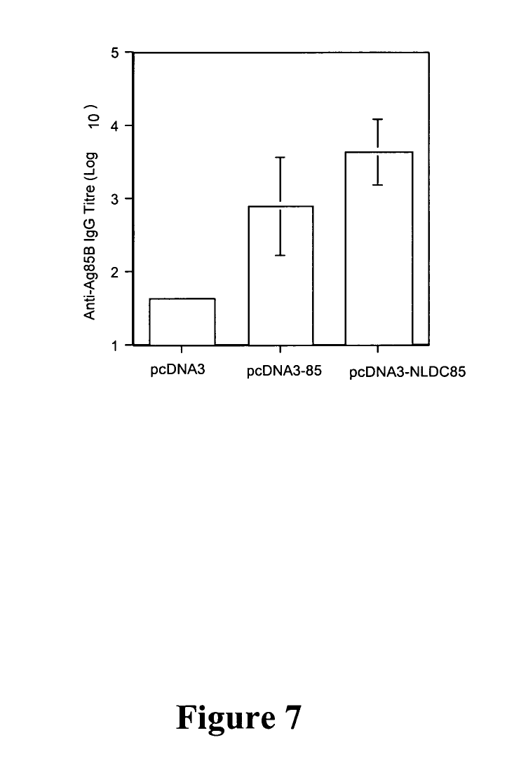

Ag85B specific antibodies in sera were assayed for by ELISA using purified protein-antigen 85B as described in Palendira et al., Infection and Immunity 70(4):1949-1956 (2002). Lymphocyte proliferation assays, ELISA and ELISPOT for IFN-γ production were conducted as previously described. Kamath et al., Infection and Immunity 67(4):1702-1707 (1999). The effects on antibody production were also tested after 1 and 2 injections to the DNA vaccines. The pcDNA3-NLDC-85B vector induced a small but significant increase in IgG antibody titer specific for antigen 85B. (FIG. 7). Two weeks after a single dose of DNA vaccine, the titer of anti-Ag85B IgG was significantly higher in the group immunized with pScNLDC-Ag85B, as compared to the group vaccinated with pAg85B, demonstrating that Ag85B targeting to DEC-205 enhanced the production of specific antibodies. In contrast, Ag85B fusion to scFv N418 did not result in enhanced immunogenicity (not shown).

These data demonstrated that targeting of the DC-specific receptor DEC-205 with an anti-DEC-205 scFv-NLDC-85B protein-antigen complex enhanced the cellular immune response to antigen 85B.

### EXAMPLE 6

### In vivo Administration of Polynucleotides Encoding scFv/NLDC145-85B and scFv/N418-85B

Polynucleotides encoding scFv/NLDC145-85B and scFv/N418-85B were administered in vivo to enhance an immune response against tuberculosis. For example, the scFv NLDC-85B DNA vaccine construct expressing the anti-DEC-205 svFv fused to the M. tuberculosis Antigen 85B (100 μg) was used to immunize C57BL6 mice (n=5) three times by the intramuscular route at two weekly intervals. Other groups of mice received pCDNA-85B expressing the mature Antigen 85B protein alone (100 μg) or the control vector pCDNA3 (100 μg) by the intramuscular route or the currently used live vaccine M. bovis BCG (Pasteur strain; 5×10⁴) once by the subcutaneous route. The mice were rested 6 weeks after the last DNA immunization or 12 weeks after the BCG vaccine. The mice were then infected, by the aerosol route, with 100 cfu of virulent Mycobacterium tuberculosis H37Rv using a Middlebrook aerosol infection apparatus (Glas-Col, Terre Haute, Ind.). The mice were sacrificed 4 weeks later and the number of organisms in the lung and spleen enumerated by culture on OADC supplemented Middlebrook 7H11 agar (Difco Laboratories).

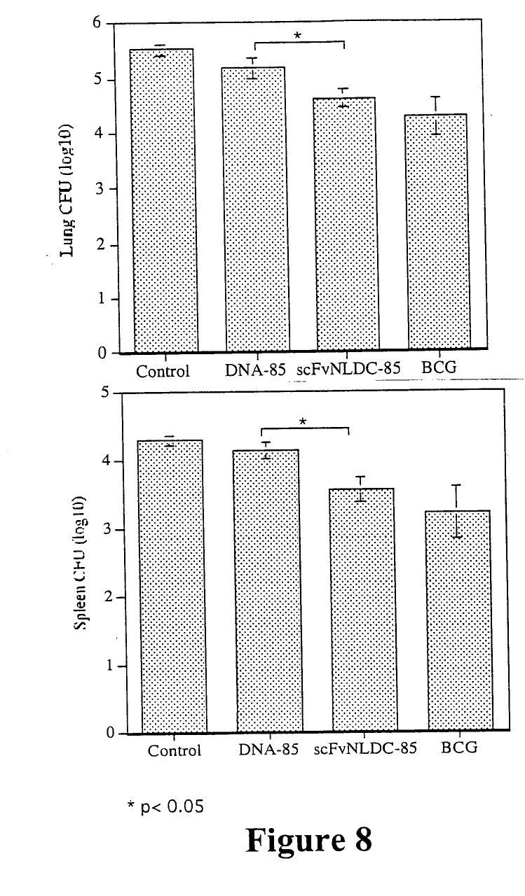

Mice immunised with the scFv NLDC-85B DNA vaccine had significantly less M. tuberculosis organisms in the lung (p<0.05) and in the spleen (p<0.05) than the recipients of the non-targeted DNA-85B (FIG. 8). There was no significant difference between the protective efficacy of the targeted scFv NLDC-85B DNA vaccine and the live vaccine BCG.

This indicates that targeting the mycobacterial protein to Dendritic Cells (DCs) with the scFv specific for the surface protein DEC-205 has increased the protective effect of DNA immunisation at the primary site of tuberculosis infection in the lung. The increase in the protective effect in the spleen indicates that this vaccine strategy has also reduced dissemination of M. tuberculosis organisms from the site of infection in the lungs to other organs. In previous studies with anti-tuberculosis subunit vaccines, immunisation with a single antigen, either as DNA or protein, had limited effect on blocking spread from the lung.

### EXAMPLE 7

### Prime/Boost Immunization Regimen Employing a PcDNA3 NLDC-85B or PcDNA3 N418-85B Prime Followed by a BCG Boost

Prior studies demonstrated that a prime/boost combination of DNA immunization prior to BCG immunization dramatically increases the effectiveness of the BCG vaccine. Accordingly, in this Example, mice are immunized with pcDNA3/scFv/NLDC-85B and pcDNA3/scFv/N418-85B vectors followed by BCG in a prime/boost regimen. Mice are immunized by intramuscular injection of 2 doses, 100 μg for each vector, or pcDNA3-85B or the negative control pcDNA3 vector. Two weeks after the second injection the same mice are immunized subcutaneously with 5×10⁴ BCG organisms. Six weeks later the protective effect is assessed by aerosol challenge with M. tuberculosis H37RB, as presented in Example 6, and the protective effect assayed 4 weeks after challenge. This prime/boost strategy using the pcDNA3/scFv/NLDC-85B and pcDNA3/scFv/N418-85B will also be testing in the guinea pig model of aerosol M. tuberculosis infection.

### EXAMPLE 8

### Targeting of scFv Tumor Antigens to Dendritic Cells Stimulates Tumor Antigen Specific T-Cell Responses

As presented in Example 5, a polynucleotide encoding scFv NLDC-85B increased T-cell and antibody responses to Mycobacterial antigen 85B. Similarly, this approach may be employed to stimulate a T-cell response against tumor antigens thereby increasing the clearance of immunologically sensitive cancers. For example, a polynucleotide encoding a scFv ovalbumin fusion protein is prepared and mice immunized with this polynucleotide, and a control polynucleotide. Following immunization, mice are challenged with tumors bearing the ovalbumin gene including the EL4 thymoma to demonstrate the effectiveness of the scFv ovalbumin fusion protein in reducing cell growth in E14 thymoma or other cells.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

What is claimed is:

1. A single-chain variable region fragment (scFv), comprising a heavy chain variable region (V_{H}) operably linked to a light chain variable region (V_{L}) wherein said scFv is capable of specifically binding to a molecule on the surface of an antigen-presenting cell (APC).

2. The scFv of claim 1 wherein said APC is a dendritic cell (DC).

3. The scFv of claim 1 wherein said molecule is selected from the group consisting of the mannose receptor (MR), chemokine receptor 1 (CCR1), B7-1 (CD80), B7-2 (CD86), CD40, CD11c, DEC-205, a Toll-like receptor (TLR), and the Fcγ receptor (FcγR).

4. The scFv of claim 3 wherein said molecule is DEC-205.

5. The scFv of claim 3 wherein said molecule is CD11c.

6. The scFv of claim 1 wherein said scFv further comprises a polypeptide linker operably linked between said V_{H} region and said V_{L} region.

7. The scFv of claim 1 wherein said scFv further comprises an affinity tag.

8. The scFv of claim 7 wherein said affinity tag comprises one or more hexahistidine.

9. The scFv of claim 1 wherein said V_{H} region and said V_{L} region are each at least 70% identical to the V_{H} and V_{L} regions of monoclonal antibody NLDC-145 disclosed herein in SEQ ID NOs: 5 and 6, respectively.

10. The scFv of claim 1 wherein said V_{H} region and said V_{L} region are each at least 90% identical to the V_{H} and V_{L} regions of monoclonal antibody NLDC-145 disclosed herein in SEQ ID NOs: 5 and 6, respectively.

11. An scFv/antigen complex, comprising an scFv of any one of claims 1-10 complexed with an antigen.

12. The scFv/antigen complex of claim 11 wherein said complex comprises a chemical crosslink between said scFv with said antigen.

13. The scFv/antigen complex of claim 11 wherein said complex comprises a fusion protein comprising said scFv and said antigen.

14. The scFv/antigen complex of claim 11 wherein said scFv further comprises an affinity tag.

15. The scFv/antigen complex of claim 14 wherein said affinity tag comprises one or more hexahistidine.

16. The scFv/antigen complex of claim 11 wherein said complex further comprises a lipid.

17. The scFv/antigen complex of claim 16 wherein said lipid is a metal-chelating lipid.

18. The scFv/antigen complex of claim 17 wherein said metal-chelating lipid is nitrilotriacetic acid ditetradecylamine.

19. The scFv/antigen complex of claim 11 wherein said antigen is from a bacterium selected from the group consisting of Mycobacterium, Chlamydia, and Ehrlichia.

20. The scFv/antigen complex of claim 19 wherein said antigen is a Mycobacterial antigen, or fragment, derivative, or variant thereof, selected from the group consisting of 85B, MPT64, and ESAT-6 as presented herein in SEQ ID NO:14, SEQ ID NO: 16, and SEQ ID NO: 18, respectively.

21. The scFv/antigen complex of claim 20 wherein said antigen is a variant of said Mycobacterial antigen 85B wherein said variant is at least about 70% identical to the sequence presented herein in SEQ ID NO: 14.

22. The scFv/antigen complex of claim 21 wherein said antigen is a variant of said Mycobacterial antigen 85B wherein said variant is at least about 90% identical to the sequence presented herein in SEQ ID NO: 14.

23. The scFv/antigen complex of claim 22 wherein said scFv/antigen complex is the scFv NLDC-145-85B presented herein in SEQ ID NO: 8, or fragment, derivative, or variant thereof.

24. The scFv/antigen complex of claim 23 wherein said scFv/antigen complex is at least about 70% identical to the scFv NLDC-145-85B presented herein in SEQ ID NO: 8.

25. The scFv/antigen complex of claim 23 wherein said scFv/antigen complex is at least about 90% identical to the scfv NLDC-145-85B presented herein in SEQ ID NO: 8.

26. The scFv/antigen complex of claim 11 further comprising a cytokine selected from the group consisting of IL-12, IL-6, IL-4, IL-1, IFNγ, GM-CSF, and TNF.

27. The scFv/antigen complex of claim 11 further comprising an inducer of a DC response to said antigen wherein said inducer is selected from the group consisting of a lipopolysaccharide (LPS) or other cell wall component, a non-methylated CpG motif, and a double-stranded RNA.

28. A fusion protein comprising an antigen-presenting cell (APC) binding protein and an antigen wherein said fusion protein is capable of specifically binding to a molecule on the surface of an APC and inducing an antigen specific T-cell response.

29. The fusion protein of claim 28 wherein said molecule on the surface of said APC is selected from the group consisting of the mannose receptor (MR), chemokine receptor 1 (CCR1), B7-1 (CD80), B7-2 (CD86), CD40, CD11c, DEC-205, a Toll-like receptor (TLR), and the Fcγ receptor (FcγR).

30. The fusion protein of claim 28 wherein said molecule on the surface of said APC is DEC-205.

31. The fusion protein of claim 28 wherein said molecule on the surface of said APC is CD11c.

32. The fusion protein of claim 28 wherein said antigen is a Mycobacterial antigen, or fragment, derivative, or variant thereof, selected from the group consisting of 85B, MPT64, and ESAT-6 as presented herein in SEQ ID NO: 14, SEQ ID NO: 16, and SEQ ID NO: 18, respectively.

33. The fusion protein of claim 28 wherein said antigen is a variant of said Mycobacterial antigen 85B wherein said variant is at least about 70% identical to the sequence presented herein in SEQ ID NO: 14.

34. The fusion protein of claim 28 wherein said antigen is a variant of said Mycobacterial antigen 85B wherein said variant is at least about 90% identical to the sequence presented herein in SEQ ID NO: 14.

35. The fusion protein of claim 28 wherein s aid antigen comprises s aid Mycobacterial antigen 85B presented herein in SEQ ID NO: 14.

36. A polynucleotide for expressing an scFv/antigen complex, comprising a first polynucleotide operably linked to a second polynucleotide wherein said first polynucleotide encodes an scFv of any one of claims 1-15 and wherein said second polynucleotide encodes an antigen.

37. The polynucleotide of claim 36 wherein said antigen is a Mycobacterial antigen, or fragment, derivative, or variant thereof, selected from the group consisting of 85B, MPT64, and ESAT-6 as presented herein in SEQ ID NO: 14, SEQ ID NO: 16, and SEQ ID NO: 18, respectively.

38. The polynucleotide of claim 37 wherein said antigen is a variant of said Mycobacterial antigen 85B wherein said variant is at least about 70% identical to the sequence presented herein in SEQ ID NO: 14.

39. The polynucleotide of claim 37 wherein said antigen is a variant of said Mycobacterial antigen 85B wherein said variant is at least about 90% identical to the sequence presented herein in SEQ ID NO: 14.

40. The polynucleotide of claim 39 wherein said scFv/antigen complex is at least about 70% identical to the scFv NLDC-145-85B presented herein in SEQ ID NO: 8.

41. The polynucleotide of claim 39 wherein said scFv/antigen complex is at least about 90% identical to the scFv NLDC-145-85B presented herein in SEQ ID NO: 8.

42. A polynucleotide comprising a polynucleotide that encodes a fusion protein of any one of claims 28-35.

43. A vector comprising a polynucleotide of any one of claims 36-42 operably linked to a transcriptional promoter.

44. A method for introducing an antigen into an antigen-presenting cell (APC) and/or a dendritic cell (DC), said method comprising the steps of:
(a) isolating from patient a sample comprising an APC and/or a DC; and
(b) contacting said APC and/or said DC with the scFv/antigen complex of claim 16,
wherein said scFv/antigen complex is in contact with said APC and/or said DC under conditions and for such time as required to permit said antigen to enter said APC and/or said DC.

45. A method for introducing an antigen into an antigen-presenting cell (APC) and/or a dendritic cell (DC), said method comprising the step of administering to said patient a composition comprising the scFv/antigen complex of claim 16.

46. A method for introducing an antigen into an antigen-presenting cell (APC) and/or a dendritic cell (DC), said method comprising the step of administering to said patient a composition comprising a polynucleotide of claim 36.

47. A method for inducing an immune response in a patient, said method comprising the steps of:
(a) obtaining from said patient a sample comprising an antigen-presenting cell (APC) and/or a dendritic cell (DC);
(b) contacting said sample with the scFv/antigen complex of claim 16 under conditions and for such a time as required to allow binding of said scFv fragment antigen complex to said APC and/or said DC; and
(c) administering said scFv/antigen APC and/or DC-bound complex to said patient.

48. A method of blocking, or substantially reducing, the activity of a target molecule on the surface of an antigen-presenting cell (APC) and/or a dendritic cell (DC), said method comprising the steps of:
(a) isolating from said patient a sample comprising an APC and/or a DC; and
(b) contacting said APC and/or said DC with the scFv of claim 1 under conditions and for such time as required to permit said binding of said scFv to said target antigen,
wherein binding of said scFv to said target molecule blocks, or substantially reduces, the activity of said target molecule.

49. The method of claim 48 wherein said target molecule is a receptor protein selected from the group consisting of the mannose receptor (MR), chemokine receptor 1 (CCR1), B7-1 (CD80), B7-2 (CD86), CD40, CD11c, DEC-205, a Toll-like receptor (TLR), and the Fcγ receptor (FcγR).
